(19) United States
(12) Patent Application Publication
(10) Pub. No.: US 2013/0312498 A1
(43) Pub. Date: Nov. 28, 2013

(54) CARDIOVASCULAR DISEASE RISK ASSESSMENT AND TREATMENT BY STEROL AND/OR STANOL MARKERS

(71) Applicant: FOUNDATION FOR HEALTH IMPROVEMENT AND TECHNOLOGY, Richmond, VA (US)

(72) Inventors: Thomas DAYSPRING, Richmond, VA (US); Joseph McCONNELL, Richmond, VA (US)

(21) Appl. No.: 13/798,538

(22) Filed: Mar. 13, 2013

Related U.S. Application Data
(60) Provisional application No. 61/651,986, filed on May 25, 2012.

(51) Int. Cl. G01N 33/18

(52) U.S. Cl. CPC G01N 33/1806
USPC 73/61.41

(57) ABSTRACT

This invention relates to a therapeutic decision diagram that determines a therapeutic guidance for achieving lipoprotein goals for a subject having various levels of cardiovascular disease risk. The therapeutic decision diagram can be used in methods to diagnose, identify or screen subjects that have, do not have or are at risk for cardiovascular disease; to differentially diagnose cardiovascular risk/disease states; to evaluate the severity or changes in severity of cardiovascular risk/disease in a subject; to monitor the efficacy of the therapies for cardiovascular disease on subjects that are undergoing such therapies; and to determine or suggest a new therapy or a change in therapy.

This application claims the benefit of U.S. Provisional Patent Application Ser. No. 61/651,986, filed May 25, 2012, which is hereby incorporated by reference in its entirety.

### FIELD OF THE INVENTION

This invention relates to a therapeutic decision diagram that determines a therapeutic guidance for achieving lipoprotein goals for a subject having various levels of cardiovascular disease risk.

### BACKGROUND

Sterols are essential components of cell membranes in animals (zoosterols, e.g., cholesterol) and plants (phytosterols). Cholesterol is essential for life, as it is a crucial membrane molecule and the precursor of steroid hormones, vitamin D, and bile acids. Production of cholesterol depends on its cellular synthesis (all cells) and absorption (enterocytes). People vary in their cholesterol balance—the amount of cholesterol they synthesize, absorb, and excrete. After dietary absorption into the enterocyte, virtually all non-cholesterol sterols and some cholesterol are effluxed back into the gut lumen via membrane sterol efflux transporters. Most humans absorb approximately 50% of the luminal sterols into the enterocyte, but hyperabsorbers absorb 60-80% and hypoabsorbers approximately 20-30%. After absorption, cholesterol, but not phytosterols, can be esterified and incorporated with triglycerides and phospholipids into the core of chylomicrons.

Phytosterols serve no physiologic function in humans or animals, and cannot be synthesized or readily absorbed by humans or animals. Because humans with normal physiology absorb very few phytosterols/stanols, their assay in blood serves as a marker of intestinal absorption. Similarly, cholesterol precursor sterols serve as synthesis biomarkers. Hyperabsorbers, in whom phytosterols do gain systemic entry, are diagnosable by increased absorption markers. With rare loss-of-function mutations in ABCGS or ABCG8, all phytosterols are absorbed and none are effluxed back out, leading to phytosterolemia, with up to 100-fold elevation in plasma phytosterol levels, associated with childhood xanthomas and premature atherosclerosis.

There have been tests measuring sterols to characterize cholesterol synthesis and cholesterol absorption events. Elevated phytosterols have been studied as having possible correlation with cardiovascular risk. However, the relationship between the cholesterol synthesis and absorption and the risks of hypercholesterolemia and cardiovascular diseases is not necessarily causal. In situations when multiple events and multiple risk factors are convoluted, the clinical information is often complicated and unclear to health practitioners; and based on such convoluted and unclear clinical messages, it is difficult for the health practitioners to predict patent's cardiovascular risk and make appropriate therapeutic guidance.

Therefore, there is a need in the art to develop a clear and concise clinical diagnostic and therapeutic decision diagram for cardiovascular risk assessment and treatment guidance to improve personalized risk assessment, and to provide more appropriate treatment decisions. This invention answers that need.

### SUMMARY OF THE INVENTION

One aspect of this invention relates to a therapeutic decision diagram that determines what therapeutic guidance for achieving lipoprotein goals, if any, should be provided to a subject having various levels of cardiovascular disease risk. This therapeutic decision diagram provides a therapeutic guidance to the subject based on risk values that comprises: (i) a first risk value determined by levels of one or more cardiovascular risk biomarkers contained in a biological sample from the subject, the cardiovascular risk biomarkers comprising at least one of low density lipoprotein particle number (LDL-P), apolipoprotein B (ApoB), or triglyceride (TG); (ii) a second risk value determined by levels of one or more cholesterol-absorption sterol and/or stanol biomarkers contained in a biological sample from the subject, and (iii) a third risk value determined by levels of one or more cholesterol-synthesis sterol and/or stanol biomarkers contained in a biological sample from the subject.

Another aspect of this invention relates to a method of prognosing, diagnosing, and/or predicting risk of cardiovascular disease in a subject. The method comprises the steps of a) measuring levels of one or more cholesterol-absorption sterol and/or stanol biomarkers contained in a biological sample from the subject, and levels of one or more cholesterol-synthesis sterol and/or stanol biomarkers contained in a biological sample from the subject; b) comparing the levels of the cholesterol-synthesis sterol and/or stanol biomarkers and the cholesterol-absorption sterol and/or stanol biomarkers to reference levels of each corresponding sterol and/or stanol biomarker to determine whether levels of the cholesterol-absorption sterol and/or stanol biomarkers are normal, decreased, or increased, and whether levels of the cholesterol-synthesis sterol and/or stanol biomarkers are normal, decreased, or increased; and c) effectuating a therapeutic plan based on the determination of the levels of the cholesterol-absorption sterol and/or stanol biomarkers and the cholesterol-synthesis sterol and/or stanol biomarkers, and an assessment of whether the subject is at high risk, moderate risk, or low risk of cardiovascular disease.

Embodiments of the invention provide methods for classifying a subject into cardiovascular disease risk categories based on the measurements of cholesterol-synthesis and cholesterol-absorption sterol and/or stanol biomarkers. Based on these measurements and categorization, specific treatment algorithm diagrams for therapeutic decisions are used to direct choice of interventional therapy. Thereafter, the risk status of the subject can be transformed by effectuating the most appropriate treatment strategy identified by the treatment algorithms.

The decision points in the therapeutic decision diagrams include a novel combination of specific analytes (cholesterol-absorption/synthesis sterol and/or stanol biomarkers) and other clinical datapoints (e.g., cardiovascular risk biomarkers such as low density lipoprotein particle number (LDL-P), apolipoprotein B (Apo-B), or triglyceride (TG)). Correlation of these cholesterol-absorption/synthesis sterol and/or stanol biomarkers with the cardiovascular risk biomarkers is a novel proxy for assessing cardiovascular risk and effectuating a therapeutic plan to achieve therapeutic goals for the cardiovascular risk biomarkers (e.g., lipoprotein goals determined by levels of LDL-P, Apo-B, etc.).

The clinical therapeutic decision diagrams provide more accurate and objective assessments for the health practitioner to optimize therapeutic decision-making Embodiments of the invention can be used as a powerful tool to improve patient management to minimize cardiovascular risk and occurrences of adverse events, including but not limited to heart attacks, atherosclerosis and strokes, and to take clinical actions in patients to improve treatment outcomes

The clinical therapeutic decision diagrams provide a clear and concise clinical diagnostic and treatment algorithm. These therapeutic decision diagrams can reduce complicated and non-obvious, multi-factorial disease processes to a simple, clear, understandable, and actionable method of diagnosis and treatment of cardiovascular risks of a subject, thus maximizing physiological recovery and minimizing risk of morbidity and mortality from cardiovascular disease in the subject. These therapeutic decision diagrams can be easily understood and interpreted, and hence be used as therapeutic guidance by health practitioners to make appropriate treatment decisions for their patients and to monitor the effectiveness of the treatments prescribed—they do not need to be an expert in the art to understand and use these therapeutic decision diagrams.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIGS. 1A-1B depict the chemical structures of sterane (FIG. 1A) and the sterol core (FIG. 1B). The carbon rings in FIG. 1A are numbered A, B, C, D from left to right.

FIG. 2 depicts the molecular structure of 3-hydroxy cholesterol.

FIG. 3 depicts the molecular structure of cholesteryl ester.

FIG. 4 depicts the molecular structure of exemplary sterols and stanols.

FIG. 5 depicts the outline of the steps involved in cholesterol synthesis.

FIG. 6 depicts the molecular structures of lathosterol (FIG. 6A) and desmosterol (FIG. 6B).

FIG. 7 depicts the major pathways in absorption and intracellular traffic of cholesterol and phytosterols in the enterocyte.

FIG. 8 shows the association between cholesterol-synthesis biomarkers and cholesterol-absorption biomarkers with prevalent cardiovascular disease (CVD) by Framingham Offspring Study (FOS).

FIG. 9 shows the relationship of simvastatin efficacy to cholesterol-absorption biomarkers by Scandinavian Simvastatin Survival Study (4S).

FIG. 10 shows the results of plant sterols in serum and plaque of carotid endarterectomy patients.

FIG. 11 shows the correlation of ezetimibe and atorvastatin administration with cholesterol-synthesis biomarkers.

FIG. 12 is an exemplary therapeutic decision diagram showing the scenarios when cholesterol-absorption and cholesterol-synthesis biomarkers in a subject are “normal,” and “normal,” respectively.

FIG. 13 is an exemplary therapeutic decision diagram showing the scenarios when cholesterol-absorption and cholesterol-synthesis biomarkers in a subject are “decreased,” and “low,” respectively.

FIG. 14 is an exemplary therapeutic decision diagram showing the scenarios when cholesterol-absorption and cholesterol-synthesis biomarkers in a subject are “decreased,” and “normal,” respectively.

FIG. 15 is an exemplary therapeutic decision diagram showing the scenarios when cholesterol-absorption and cholesterol-synthesis biomarkers in a subject are “decreased,” and “increased,” respectively.

FIG. 16 is an exemplary therapeutic decision diagram showing the scenarios when cholesterol-absorption and cholesterol-synthesis biomarkers in a subject are “increased,” and “normal,” respectively.

FIG. 17 is an exemplary therapeutic decision diagram showing the scenarios when cholesterol-absorption and cholesterol-synthesis biomarkers in a subject are “increased,” and “increased,” respectively.

FIG. 18 is an exemplary therapeutic decision diagram showing the scenarios when cholesterol-absorption and cholesterol-synthesis biomarkers in a subject are “increased,” and “decreased,” respectively.

FIG. 19 is an exemplary therapeutic decision diagram showing the scenarios when cholesterol-absorption and cholesterol-synthesis biomarkers in a subject are “normal,” and “low,” respectively.

FIG. 20 is an exemplary therapeutic decision diagram showing the scenarios when cholesterol-absorption and cholesterol-synthesis biomarkers in a subject are “normal,” and “increased,” respectively.

### DETAILED DESCRIPTION OF THE INVENTION

### Sterols/Stanols

A polyphenol is a mutliphenol compound typically found in plants.

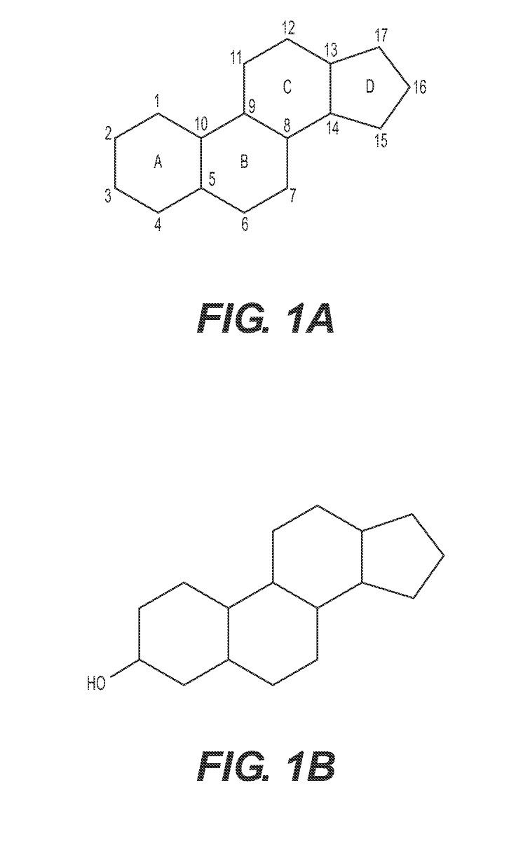

Steroids and sterols are derivatives of sterane (cyclopentanoperhydrophenanthrene), which is a class of 4-cyclic compounds that constitutes the core of sterols and steroids (see FIG. 1A).

Sterols are also known as steroid alcohols, because of their hydroxy group at the third position of the A ring (see FIG. 1B). The nonpolar hydrocarbon tail and polar hydroxy group convey critical amphipathic properties that allow proper alignment in lipid membrane mono- and bilayers.³ Sterols include zoosterols (animals) and phytosterols (plants). They are essential components of cell membranes in animals and plants.

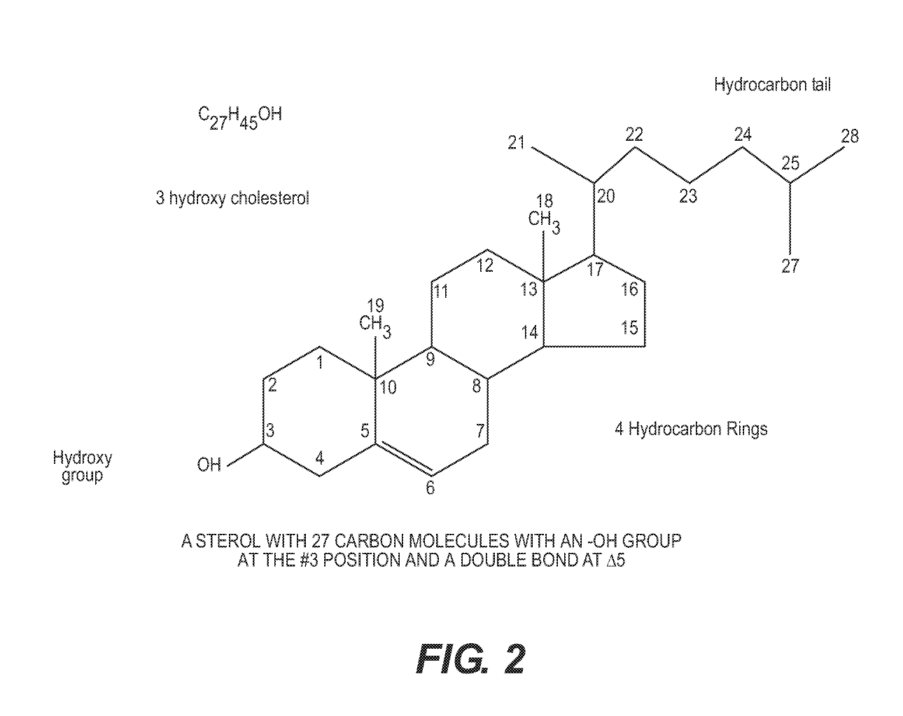

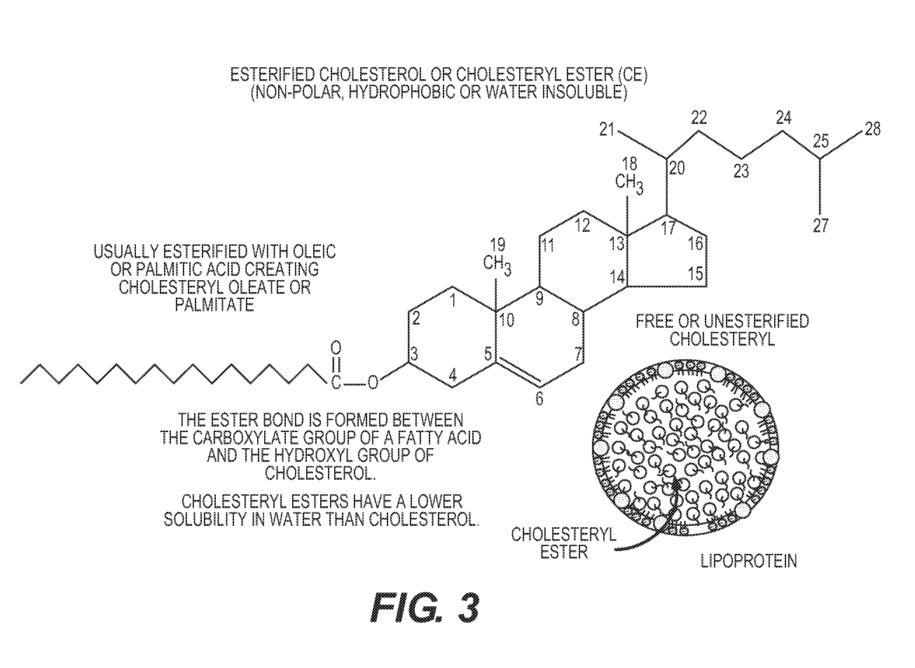

The predominant zoosterol is cholesterol. Sterols that have structural similarity to cholesterol are referred to as non-cholesterol sterols^{1,2}. Production of cholesterol depends on its cellular synthesis (virtually every cell in the body) and absorption (enterocytes) (see FIG. 2). 3-hydroxy cholesterol is also called free or unesterified cholesterol (UC) and it is the active form of cholesterol which can be converted to sterol derivatives such as hormones or bile acids. The majority of cholesterol present in humans exists as an inactive ester, namely cholesteryl ester (CE), a nonpolar or hydrophobic molecule which serves as the storage or transportation form of cholesterol (FIG. 3). Esterification can occur in cells catalyzed by acyl-cholesterol acyl transferase (ACAT) or within lipoproteins catalyzed by lecithin cholesterol acyl-transferase (LCAT). Phytosterols, on the other hand, are poor substrates for human ACAT and LCAT.

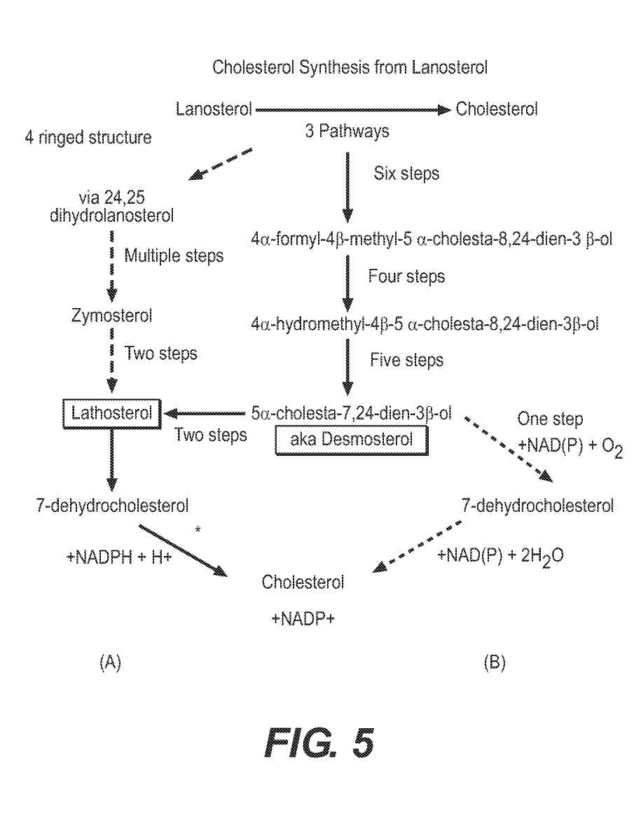

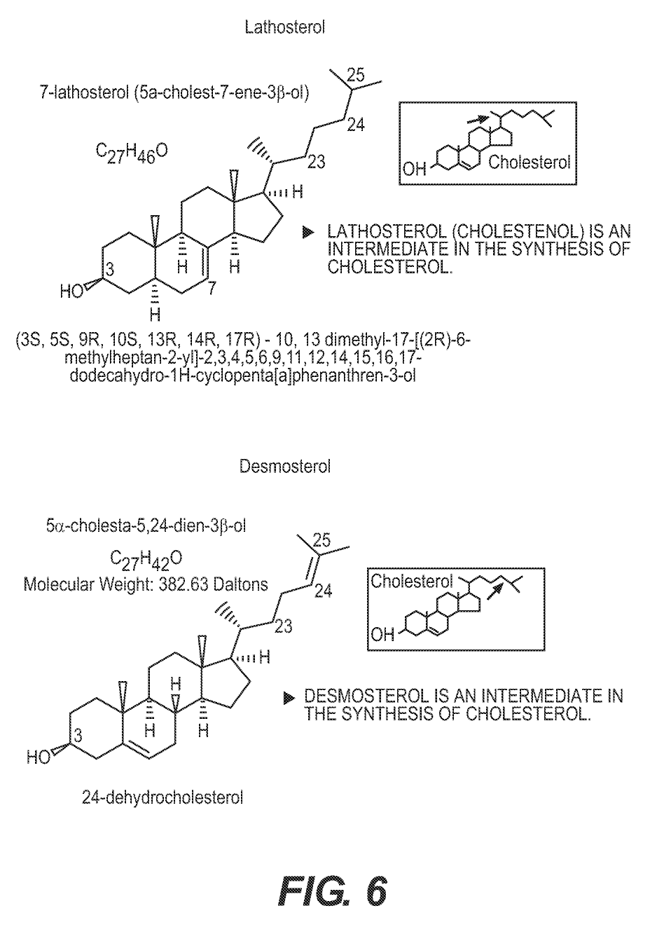

Cholesterol is synthesized from acetate and acetoacetyl-CoA in a complex 37-step process, utilizing multiple enzymes (see FIG. 5). Some of the intermediary sterols in the synthetic chain are squalene, lathosterol and desmosterol (see FIG. 6), measurements of which can serve as a marker of cholesterol synthesis. The human diet includes many exogenous sterols from plants (e.g., sitosterol, campesterol, and stigmasterol), animals (e.g., cholesterol), shellfish sources (e.g., desmosterol, and fucosterol) and yeast sources.

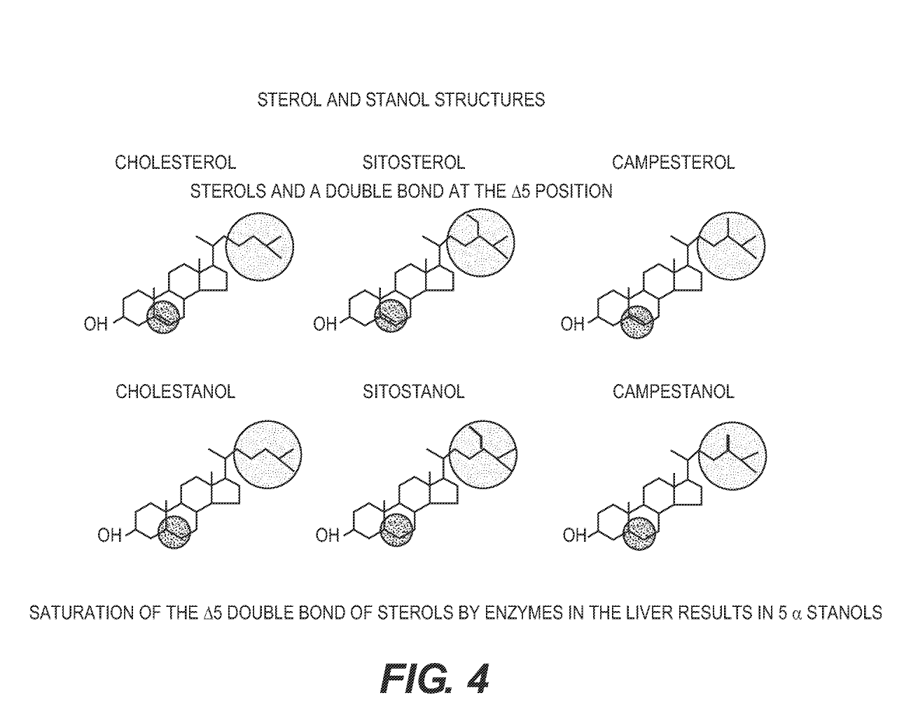

There are over forty different plant sterols (or phytosterols). Phytosterols are similar in structure to cholesterol, but have methyl, ethyl or other groups in their aliphatic side chains (see FIG. 4). These differences minimize their absorption compared to cholesterol. Sitosterol represents 80% of non-cholesterol sterols in the diet^{4,5}.

Each of these sterols, as well as others known to one skilled in the art, falls under the definition of “sterol” for the purposes of this invention.

Stanols are simply saturated sterols where the Δ5 double bond present in the sterol is hydrogenated, further impairing absorption (see FIG. 4)^{1,6}. For instance, the stanol metabolite of cholesterol is called cholestanol; and the stanol metabolite of sitosterol is sitostanol.

Cholestanol is present in food substances, especially meats, or is produced when intestinal bacteria metabolize cholesterol (e.g., of endogenous (biliary) or exogenous origin). Stanols are not readily absorbed, and hence cholestanol can be used as a marker of sterol absorption. Additionally, a rare enzyme deficiency called cerebrotendinous xanthomatosis (CTX) causes marked elevation of cholestanol, leading to a specific lipidosis (cerebrotendinous xanthomatosis), xanthomata and CNS neurologic abnormalities.¹⁵

Each of these stanols, as well as others known to one skilled in the art, falls under the definition of “sterol and/or stanol” biomarker for the purposes of this invention, particularly when being used to diagnose or predict risk of cardiovascular disease.

Therapeutically, there may be differences between using sterols and stanols to lower levels of cholesterol. Distinction should be made according to the method described herein whether to administer sterols versus stanols to patients in modulating the levels of cholesterol in patients.

Plant sterols and stanols can be incorporated into margarines or other food products if they are commercially esterified (i.e., combined with fatty acids). Many esterified phytosterols can be available, but sitostanol currently is the only commercial stanol (known as a Benecol).⁷

### Physiology

The human diet includes UC, CE, phytosterols, and to a lesser degree some stanols. Intestinal esterolases convert some of the ingested CE into UC. However, after a meal, the vast majority of the UC in the jejunum is of biliary origin. Moreover, some intestinal cholesterols are also converted by microbes into cholestanol or coprostanol for excretion in stool. All of the lipids in the gut lumen are collectively organized and emulsified by lecithin (phosphocholine), a phospholipid in biliary secretions. The lipids are then surrounded by amphipathic bile acids into mixed biliary micelles which contains collections of UC, phytosterols, stanols, phospholipids, monoacylglycerols, and fatty acids. The micelles “ferry” these lipids to the epithelium of the intestinal microvilli. Fatty acids are then absorbed through the lipid cell membranes into enterocytes by passive diffusion or membrane-located fatty acid transport proteins.

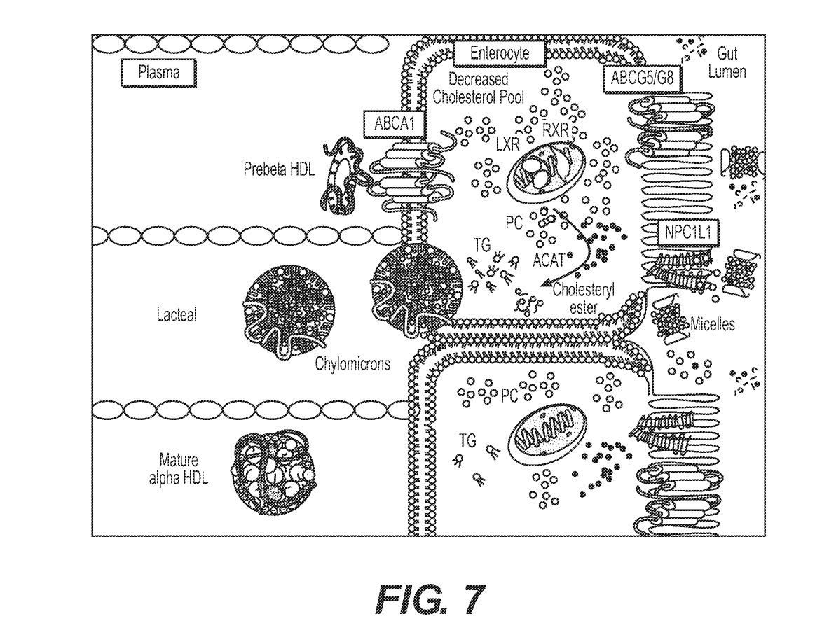

The unesterified sterols (but not stanols) in the micelles are internalized into the enterocyte via a sterol permease (a protein involved with absorption of sterols) named as the Niemann Pick C1 Like 1 (NPC1L1) protein, which utilizes other proteins (AP2-clathrin) to facilitate sterol absorption (see FIG. 7). NPC1L1, partially regulated by PPAR-α and -Δ, is expressed in both the brush border of the intestinal epithelium and at the hepatobiliary cell junction. NPC1L1 is not involved with fatty acid absorption. Most humans absorb about 50% of the sterols in the gut, but some people are hyperabsorbers (absorbing as much as 60-80%) or hypoabsorbers (absorbing only ˜20-40%).⁸ NPC1L1 expressed at the hepatobiliary interface facilitates re-entry of biliary UC back into the liver. Cholesteryl ester cannot pass through NPC1L1 and thus is not absorbed unless it is hydrolyzed.

Once the sterols gain entry into the enterocytes, several pathways exist for their utilization or disposal:

1) Unesterified cholesterol (but not phytosterols) is a substrate for acyl-cholesterol acyl transferase 2 (ACAT2) which leads to the production of the hydrophobic molecule CE. Cholesteryl ester and enterocyte-produced triglycerides (TG), with the aid of microsomal TG transfer protein (MTP), are lipidated to apolipoprotein B48 (apoB48), resulting in chylomicron production (see FIG. 7).

2) Unesterified cholesterol mixes with phospholipids to form the chylomicron surface. UC can also be effluxed via ATP binding cassette transporters A1 (ABCA1) into apolipoprotein A-I (apoA-I) or prebeta HDLs (see FIG. 7). ApoE can also serve as an UC acceptor.

3) Excess UC and most phytosterols are effluxed back to the gut lumen via ABCGS and ABCG8 (see FIG. 7). Any phytosterols that reach the liver are rapidly effluxed into the bile, via ABCGS and ABCG8 at the hepatobiliary junction, for delivery to the gut and fecal elimination. If there is reduced expression of hepatic ABCGS/G8, the amphipathic phytosterols will be incorporated into the surface of very low density lipoproteins (VLDLs) and ultimately LDLs where they can gain arterial entry and promote atherogenesis.

4) The inability to be esterified by ACAT promotes phytosterol return to the gut lumen and markedly restricts their systemic absorption. If there are defects in ABCGS and/or ABCG8 expression, phytosterols are not rapidly returned to the gut lumen and can be incorporated into the surface layers of chylomicrons and, via ABCA1, into enterocyte lipidated HDLs.¹⁰

Thus evolution, by making phytosterols poor substrates for ACAT2, and via placement of ABCG5 and ABCG8 in enterocytes and hepatocytes, has gone to great lengths to keep phytosterols out of the human body, leading to speculation that these molecules not only may be unnecessary but might be even toxic.^{2,9} Indeed, homozygous absence of these sterol efflux proteins results in the disease sitosterolemia or phytosterolemia which is associated with xanthomata and premature atherosclerosis.¹¹ It is speculated that if noncholesterol sterols (which cannot be esterified) gain entry into the arterial wall, the exposure of the 3-hydroxy group makes them more susceptible to oxidation than CE, and may facilitate foam cell formation.¹² Thus, health practitioners should be cautious when they recommend dietary phytosterol supplementation as an adjunctive cholesterol-lowering therapy. The 2001 European Guidelines (EAS/ECS) recommend close monitoring when such phytosterol products are prescribed.¹³

The terminology of “sterol-absorption” may be understood differently in different context. When health practitioners refer to a substance being absorbed, they typically visualize such molecules moving from the gut lumen into the plasma. However, when experts refer to sterol absorption in the intestinal enterocyte, they are referring to the process whereby the sterol enters the enterocyte by traversing the luminal cell membrane, not to whether it reaches the plasma inside a chylomicron. The enterocyte-internalized (absorbed) sterol may or may not get into the plasma because the enterocyte has the ability, using membrane protein efflux transporters, to return sterol molecules to the gut lumen. Thus, not all absorbed sterols necessarily get into the plasma or lymphatic system. Entry of sterols into the systemic circulation is a result of the interplay between sterol influx (NPC1L1), sterol esterification by ACAT2, and sterol efflux transporters (ABCA1, ABCG5 and ABCG8). Sterol homeostasis is rendered even more complex by the presence of these same transporters at the hepatobiliary interface. Accordingly, both enterocytes and hepatocytes can respectively absorb sterols from the gut lumen (into enterocytes) and bile (into hepatocytes), or export sterols from enterocytes or hepatocytes into the gut lumen or bile.

The rate at which persons absorb cholesterol is variable and depends on the many cellular nuclear transcription factors that regulate cholesterol homeostasis, including the sterol regulatory element binding proteins (SREBPs), liver X receptors (LXRs), farnesoid receptors (FXRs), and peroxisome proliferator-activated receptors (PPARs) alpha and delta. If the body needs cholesterol there will be an increase in both cholesterol synthesis and absorption: In both the liver and intestine there will be up-regulation of NPC1L1 and down-regulation of ABCG5 and ABCG8. On the contrary, in cholesterol overload situations the opposite will occur, namely a down-regulation of NPC1L1 and up-regulation of ABCG5 and ABCG8.¹⁴

### Clinical Interpretations

Because cholesterol is produced by every cell, cholesterol measurements do not provide information regarding its origin, i.e., whether an elevated cholesterol level is the result of increased absorption, increased endogenous synthesis, or decreased clearance. Humans normally absorb very few phytosterols. Thus, phytosterol assay in blood serves as a marker of intestinal absorption. An elevated plasma level of sitosterol or campesterol indicates a hyperabsorption state and individuals with phytosterolemia have marked elevations of these markers.¹¹

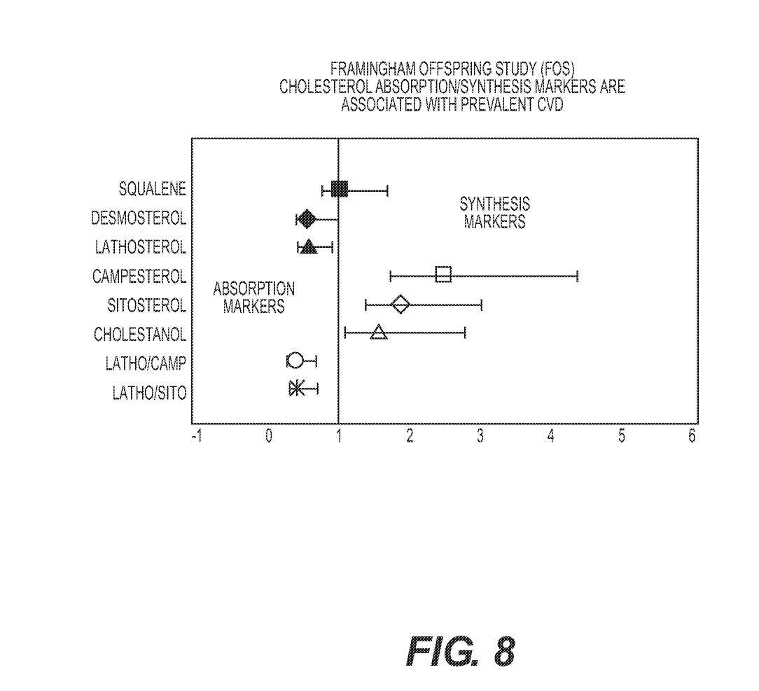

Several trials including the Framingham Offspring, PROCAM (Prospective Cardiovascular Munster) study, Helsinki Business Study, LURIC (Ludwigshafen Risk and Cardiovascular Health) study, and the Cardiovascular Risk in Young Finns Study have shown that markers of hyperabsorption (sitosterol, campesterol or cholestanol) are useful indicators of CHD risk (see FIG. 8).¹⁶⁻²² In hyperabsorptive states, as the liver receives increased chylomicron delivery of phytosterols, UC and CE, there will be an LXR-mediated down-regulation in the production of HMGCoA reductase, which slows cholesterol synthesis (this can be confirmed by finding low plasma lathosterol or desmosterol levels). Indeed, with respect to markers of synthesis (lathosterol or desmosterol), either elevated levels (as in familial hypercholesterolemia (FH) or some apoE disorders) or low levels in the presence of elevated absorption markers, signify increased risk. There are trials such as the Dallas Heart Study that have not found an association between absorption markers and increased cardiovascular risk.^{18,24}

The DEBATE study (Drugs and Evidenced Based Medicine in the Elderly Study²⁵) showed that even with the metabolic syndrome, low cholesterol absorption was associated with fewer recurrent CVD events and higher survival rates in the elderly, perhaps due to a lower lifetime cholesterol exposure.²⁵

Not everyone has perfectly functioning ABCG5/G8 transporters. Studies have shown that noncholesterol sterols do gain systemic entry in some people.^{26,27} Patients with up-regulation of NPC1L1 and/or down-regulation of ABCG5 or ABCG8 will have elevated sterol absorption markers. Common hyperabsorptive states include patients with a strong family history of premature CHD, postmenopausal women, patients with T2DM, patients using statins, and in some, but not all studies, men with the apoE4 allele hyperabsorb cholesterol.²⁸⁻³¹ Phytosterols have been found in carotid plaque in statin users.³² Such patients have severe, moderate or slightly elevated absorption markers (cholestanol, sitosterol, or campesterol). The plasma phytosterol levels are the highest in the phytosterolemia patients (an autosomal recessive disorder; complete absence of ABCG5 or ABCG8 caused by a homozygous mutation in the underlying gene).¹¹ Health practitioners would not know which patients may be over absorbing cholesterol or noncholesterol sterols without measuring markers of absorption. Such knowledge can influence both lifestyle counseling and lipid drug choice.

### Treatment Consideration

Some studies have shown that measurement of sterol absorption and synthesis markers has the potential to help a health practitioner choose effective therapies.

A health practitioner may generally refer to any individual that is trained to provide health care services, including, but are not limited to, a physician, physician assistant, nurse, midwife, dietitian, therapist, psychologist, pharmacist, clinical officer, phlebotomist, emergency medical technician, medical laboratory scientist, medical prosthetic technician, social worker, community health worker, and a wide variety of other human resource trained to provide some type of health care service. Health practitioners can work in hospitals, health care centers, or other service delivery points, including care and treatment services in private homes; or in academic training, research, and administration.

Hyperabsorptive states, statin-induced or not, can be treated with the use of supplements (i.e., plant stanols) or drugs that reduce absorption (ezetimibe or fibrates).³³⁻³⁶ Cholesterol hypersynthetic states can be treated with lifestyle and statins. Instances of elevated apoB or LDL-P occurring with normal absorption and synthesis markers suggest decreased clearance or increased production of apo-B particles or decreased clearance of atherogenic lipoproteins, and they can be treated respectively with medications that up-regulate LDL receptors (statins, ezetimibe, bile acid sequestrants) or medications that reduce apoB particle production.

Statins inhibit the action of HMGCoA reductase, the rate limiting enzyme of the cholesterol synthesis pathway. Plasma levels of markers of cholesterol synthesis (desmosterol, lathosterol) will be reduced by statins. Reflexively, statin-inhibition of cholesterol synthesis induces up-regulation of the NPC1L1 protein which will influx UC from bile to liver and UC and phytosterols from the intestinal lumen to enterocyte. Thus, statins often increase sitosterol, campesterol and cholestanol levels.²⁹

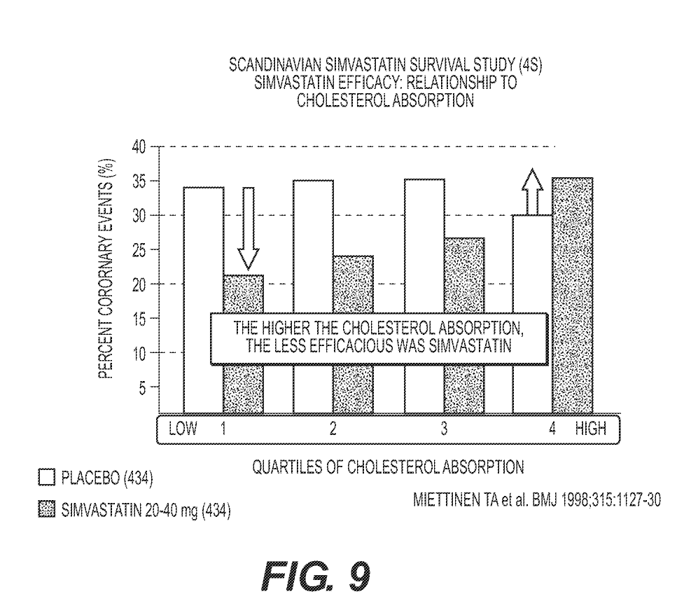

Typically, health practitioners prescribe statins to patients with elevated LDL-C. However, if high LDL-C is due not to increased synthesis, but rather increased absorption (which results in decreased HMGCoA reductase activity), the statin will be ineffective. Indeed, as demonstrated in the 4S trial, simvastatin had no effect on CV endpoints when administered to hyperabsorbers of cholesterol (identified by increased cholestanol levels; see FIG. 9).³⁷

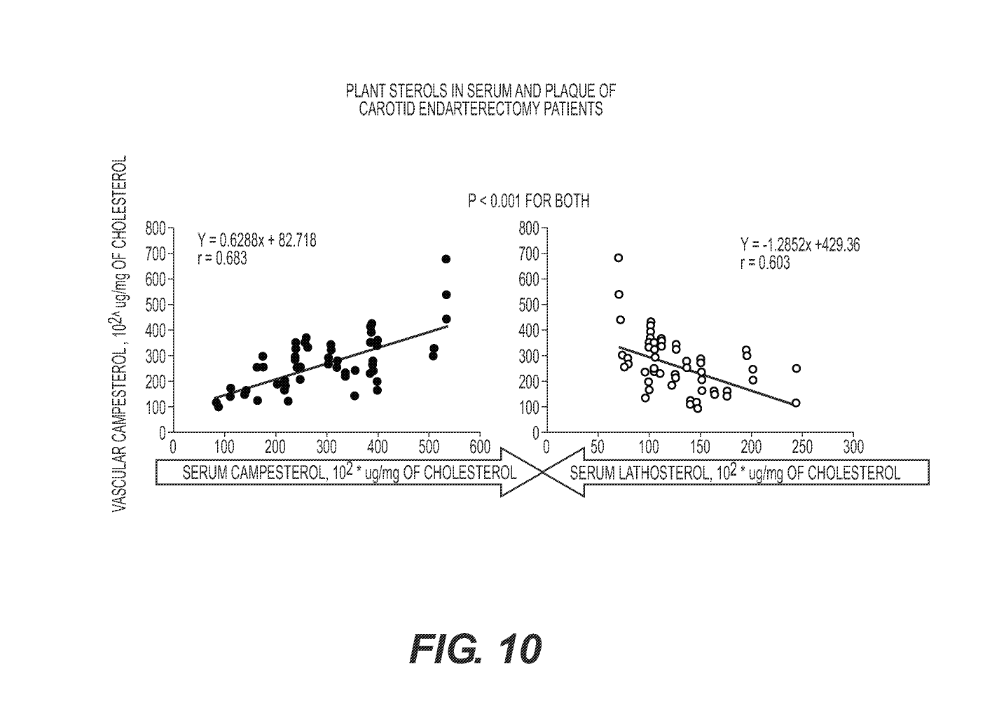

Thus, using ezetimibe would likely have helped such patients achieve LDL-C goals more effectively than the statin monotherapy. Of perhaps greater concern is the trial which showed that statin-treated patients, but not drug-naive patients, undergoing carotid endarterectomy plaque analysis showed increased plaque campesterol and decreased cholesterol (see FIG. 10).³²

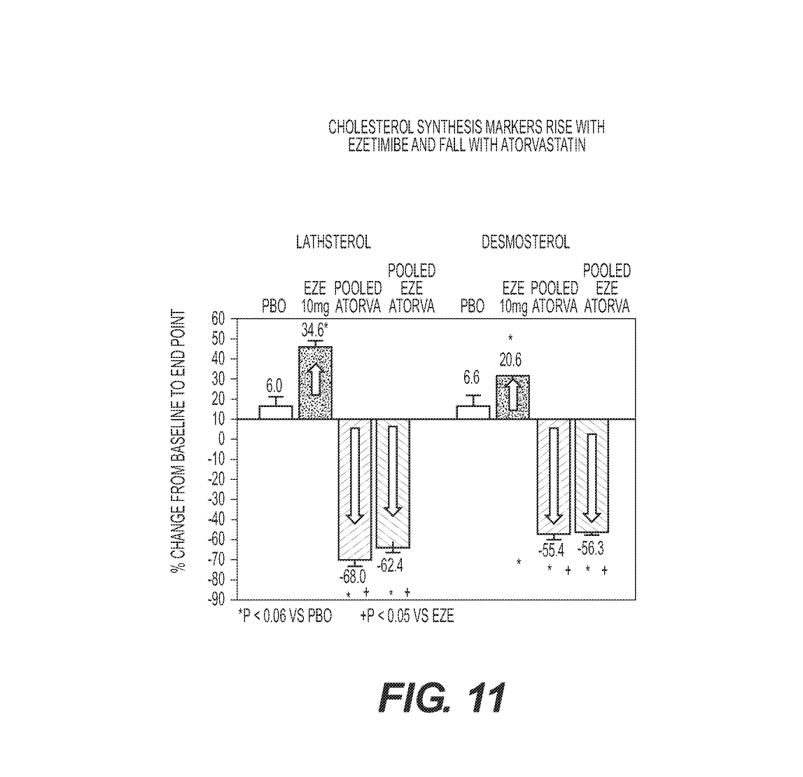

This implies that the statin-induced over-absorption of phytosterols resulted in those sterols-entering plaque. Numerous trials show that the use of statin monotherapy, especially at the high doses, although reducing cholesterol, may significantly increase intestinal, biliary and plasma phytosterol levels (by up-regulating NPC1L1). In this regard, atorvastatin at the 80 mg dose seems to be a particularly potent undesirable offender (see FIG. 11).³⁸

Ezetimibe (e.g., Zetia®) blocks sterol absorption from micelles by interfering with (by binding to) the NPC1L1/AP2-clathrin complex in the intestinal epithelium.^{34,39} Ezetimibe typically reduces sterol absorption by about 50%. Because ezetimibe blocks the absorption of all sterols, it is approved not only to reduce cholesterol levels but also to reduce the very high noncholesterol sterol levels seen in patients with phytosterolemia (sitosterolemia). Since the vast majority of intestinal UC is of biliary, not exogenous origin, ezetimibe in effect has only a minor effect on blocking the absorption of ingested cholesterol. NPC1L1 is also expressed at the hepatobiliary interface and thus facilitates re-entry of biliary cholesterol back into the liver. Ezetimibe monotherapy will reduce chylomicron delivery of cholesterol to the liver. Thus, ezetimibe monotherapy, by inhibiting cholesterol absorption and reflexively increasing cholesterol synthesis, will reduce markers of absorption (sitosterol, campesterol, cholestanol) but increase synthesis markers (desmosterol, lathosterol).³⁴ One solution is to use a low dose statin with the ezetimibe, so that both synthesis and absorption markers will be normalized. Ezetimibe has no effect on fatty acid absorption.^{40,41}

Fibrates are peroxisome proliferator-activated receptor (PPAR)-alpha agonists and have a multitude of effects on lipid and lipoprotein levels. PPAR-alpha is one of the nuclear transcription factors that regulate expression of NPC1L1. By down-regulating NPC1L1, fibrates reduce cholesterol absorption and enhance its excretion in the stool.³³ They have been a mainstay of treatment for those with phytosterolemia for some time. Ezetimibe interferes with NPC1L1 function. Combining fenofibrate with ezetimibe (an on-label use) results in a significant inhibition of sterol absorption and enhanced lowering of both LDL-C and non-HDL-C.⁴²

If plant sterols and stanols are esterified (combined with fatty acids), they can be incorporated into margarines or other products. By competing with cholesterol for entry into biliary micelles, they reduce the amount of cholesterol that is available for internalization by NPC1L1. By themselves, or when combined with statins, ezetimibe, colesevelam or fibrates, such products can enhance LDL-C lowering.^{1,7,43} However, phytosterols (but not phytostanols) are not desirable when the subject already has elevated phytosterol levels.

Accordingly, one aspect of this invention relates to a therapeutic decision diagram that determines what therapeutic guidance for achieving lipoprotein goals, if any, should be provided to a subject having various levels of cardiovascular disease risk. This therapeutic decision diagram providing a therapeutic guidance to the subject based on risk values comprises: (i) a first risk value determined by levels of one or more cardiovascular risk biomarkers contained in a biological sample from the subject, the cardiovascular risk biomarkers comprising at least one of low density lipoprotein particle number (LDL-P), apolipoprotein B (ApoB), or triglyceride (TG); (ii) a second risk value determined by levels of one or more cholesterol-absorption sterol and/or stanol biomarkers contained in a biological sample from the subject, and (iii) a third risk value determined by levels of one or more cholesterol-synthesis sterol and/or stanol biomarkers contained in a biological sample from the subject.

The term “subject” as used herein includes, without limitation, mammals, such as humans or non-human animals. Non-human animals may include non-human primates, farm animals, sports animals, rodents or pets. A typical subject is human and may be referred to as a patient. Mammals other than humans can be advantageously used as subjects that represent animal models of the cardiovascular disease or for veterinarian applications.

A “biological sample” encompasses a variety of sample types obtained from a subject with a biological origin. Typically used here is a biological fluid sample including, but not limited to, blood, cerebral spinal fluid (CSF), interstitial fluid, urine, sputum, saliva, mucous, stool, lymphatic, or any other secretion, excretion, or and other bodily liquid samples. Exemplary biological fluid sample can be a blood component such as plasma, serum, red blood cells, whole blood, platelets, white blood cells, or components or mixtures thereof. Suitable biological samples also include samples that have been manipulated in any way after their procurement, such as by treatment with reagents, solubilization, purification, or extraction or enrichment of certain components, such as sterols/stanols.

Biomarkers used herein include conventional cardiovascular risk biomarkers that are typically used by health practitioners, such as low density lipoprotein particle number (LDL-P), apolipoprotein B (ApoB), triglyceride (TG), or low density lipoprotein cholesterol (LDL-C). For each of these cardiovascular risk biomarkers, the level of the biomarker can be measured and compared to a reference level, to preliminarily categorize whether the subject has a high risk, moderate risk, or low risk of cardiovascular disease. Each of these cardiovascular risk biomarkers can be used individually or combined together to determine the first risk value.

Many other biomarkers and factors can be used to determine the first risk value, i.e., preliminary categorization of the high risk, moderate risk, or low risk of cardiovascular disease for a subject, as known by one skilled in the art. Additional biomarkers may include high density lipoprotein cholesterol (HDL-C), high-sensitivity C-reactive protein (hs-CRP), lipoprotein A (Lp(a); apoA-I or HDL), etc. Suitable factors to determine the first risk value may also include age, gender, high blood pressure, high serum cholesterol levels, tobacco smoking, excessive alcohol consumption, premature family history, obesity, lack of physical activity, psychosocial factors, diabetes mellitus, air pollution, etc.

Determinations of the first risk value, i.e., preliminary categorization of whether the subject has a high risk, moderate risk, or low risk of cardiovascular disease based on various conventional cardiovascular risk biomarkers and factors, do not require uniform reference values or reference ranges for each of these biomarkers and factors—the determinations are subjected to a health practitioner's discretion, based on their knowledge and empirical experiences.

Biomarkers used herein also include the cholesterol-synthesis sterol and/or stanol biomarkers and the cholesterol-absorption sterols/stanols biomarkers. Cholesterol precursor sterols typically serve as biomarkers of cholesterol synthesis, and phytosterols typically serve as biomarkers of cholesterol absorption. Exemplary cholesterol-absorption sterol and/or stanol biomarkers include campesterol, sitosterol (e.g., β-sitosterol), and cholestanol. Exemplary cholesterol-synthesis sterol and/or stanol biomarkers include desmosterol, lathosterol, and squalene. Each of the cholesterol-absorption sterols/stanols biomarkers can be used individually or combined with another cholesterol-absorption sterols/stanols biomarker to determine the second risk value. Each of the cholesterol-synthesis sterols/stanols biomarkers can be used individually or combined with another cholesterol-synthesis sterols/stanols biomarker to determine the third risk value.

These biomarkers from a subject can be measured, detected and analyzed using various assays, methods and detection systems known to one of skill in the art. Methods to measure or detect levels of biomarkers include, but are not limited to, mass spectrometry (MS), gas chromatography (GC), liquid chromatography (LC), matrix-assisted laser desorption ionization-time of flight (MALDI-TOF), ion spray spectroscopy, ultra-violet spectroscopy (UV-vis), fluorescence analysis, radiochemical analysis, near-infrared spectroscopy (near-IR), infrared (IR) spectroscopy, nuclear magnetic resonance spectroscopy (NMR), light scattering analysis (LS), and combinations thereof. For instance, a rapid and high-throughput measurement and analysis of sterols/stanols or derivatives using liquid chromatography tandem mass spectrometry (LC-MS/MS) has been described in detail in U.S. Provisional Application No. 61/696,613, entitled, “Rapid and High-throuput Analysis of Sterols/stanols or Derivatives Thereof,” filed Sep. 4, 2012, which is herein incorporated by reference in its entirety.

The term “measure” refers to a quantitative or qualitative determination of the amount or concentration of a molecule or a substance. The term “level,” “amount,” or “concentration” can refer to an absolute or relative quantity. The level of each biomarker can be compared to a reference level of the corresponding biomarker, and the difference, if any, in the measured level of the biomarker in the subject compared to the reference level is then identified. This difference is used to determine the risk value or risk category as described herein. For example, the level of each cholesterol-absorption sterol and/or stanol biomarker can be compared to a reference level of the corresponding sterol and/or stanol biomarker to determine whether the second risk value is normal, decreased, or increased risk of cardiovascular disease. Similarly, the level of each cholesterol-synthesis sterol and/or stanol biomarker can be compared to a reference level of the corresponding sterol and/or stanol biomarker to determine whether the third risk value is normal, decreased, or increased risk of cardiovascular disease. The level of the biomarker can be an absolute quantity or a relative value, e.g., a ratio adjusted relative to quantity of cholesterol.

As used herein, a “reference value” or “reference level” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the subject being tested, but at an earlier point in time. The reference value can be based on a large number of samples, such as from population of healthy subjects, or based on a pool of samples including or excluding the sample to be tested.

In one embodiment, reference ranges for cholesterol-absorption biomarkers and cholesterol-synthesis biomarkers have been developed empirically via clinical studies as shown below in Table 1. These reference ranges include both the ranges of absolute reference levels of the biomarkers and the ranges of relative reference levels, using a ratio of the quantity of the biomarker to the quantity of cholesterol, for each biomarker. Hyper-absorber, normal-absorber, hypo-absorber, hyper-synthesizer, normal-synthesizer, and hypo-synthesizer phenotypes are classified by the empirical ranges listed in the table, based on quintile cut-points from a 500 patient study run by HDL.

TABLE 1
Clinical reference ranges of cholesterol-synthesis
biomarkers and cholesterol-absorption biomarkers
determined empirically via clinical studies *.
STEROLS

| | Hyper | Normal | Hypo |
|---|---|---|---|
| Absorption Marker | | | |
| Campesterol(μg/mL) | >4.43 | 2.1-4.4 | <2.1 |
| Campesterol ratio 10² | >239.7 | 114.5-239.7 | <114.5 |
| mmol/mol cholesterol | | | |
| Sitosterol(μg/mL) | >3.17 | 1.43-3.17 | <1.43 |
| Sitosterol ratio 10² | >167.8 | 75.8-167.8 | <75.8 |
| mmol/mol cholesterol | | | |
| Cholestanol (μg/mL) | >3.4 | 2.0-3.4 | <2.0 |
| Cholestanol ratio 10² | >194.2 | 116.8-194.2 | <116.8 |
| mmol/mol cholesterol | | | |
| Synthesis Marker | | | |
| Desmosterol(μg/mL) | >1.27 | 0.5-1.27 | <0.5 |
| Desmosterol ratio 10² | >64.4 | 31.0-64.4 | <31.2 |
| mmol/mol cholesterol | | | |

* In this application, all references to “hyper-”, “normal” and “hypo-” refer to the values referenced in this table.

The reference ranges for cholesterol-absorption biomarkers and cholesterol-synthesis biomarkers can assist health practitioners identify subjects with increased or decreased risks of phytosterolemia and/or atherosclerosis. Health practitioners can predict relative risk of atherosclerosis by observing the phytosterol levels in the patients. However, health practitioners can not use phytosterol levels as absolute markers of atherosclerosis (i.e., health practitioners can not label a subject as having phytosterolemia when there is small increase of phytosterol level in the subject), unless the level of campesterol or sitosterol in a phytosterolemic patient is within the range of 100-300 μg/mL.

Based on the above measurements and analyses, the subject can be categorized in accordance with the first, second and third risk values. The first risk value, measuring high risk, moderate risk, or low risk of cardiovascular disease using the conventional cardiovascular risk biomarkers and factors, is the primary decision point. Each of these risk categories determined in the first risk value carries a different (or desirable) lipoprotein goal (e.g., desirable levels of LDL-P and/or apoB) of therapy. The second and third risk values, based on measurements and categorization of cholesterol-absorption biomarkers and cholesterol-synthesis biomarkers, are correlated with the first risk value to reach specific treatment algorithm diagrams for therapeutic decisions, which are used to direct choice of interventional therapy. Thereafter, the risk status of the subject can be transformed by effectuating the appropriate lipoprotein treatment strategy identified by the treatment algorithms.

Each risk category (e.g., categories of high, medium, or low risk associated with the first risk value; and similarly for the second and third risk value) may be associated with one or more biomarker chosen by the health practitioner. The categories, particularly for the first risk value, may be derived from the current literature or according to the findings of health practitioner by his/her discretion. For example, it is known in the art that an individual with a serum/plasma concentration of apoB that is greater than 120 mg/dL and/or a serum/plasma concentration of LDL-P that is greater than 1600 nmol/L has a high risk for cardiovascular disease; while an individual with a serum/plasma concentration of apoB that is greater than 90 mg/dL but less than 120 mg/dL and/or a serum/plasma concentration of LDL-P that is greater than 1200 nmol/L but less than 1600 nmol/L has a medium risk for cardiovascular disease; and an individual with a serum/plasma concentration of apoB that is lower than 80 mg/dL and/or a serum/plasma concentration of LDL-P that is lower than 1000 nmol/L has a low risk for cardiovascular disease. It is also known in the art that when an individual has a serum/plasma concentration of TG that is greater than 500 mg/dL, that individual has a high risk for cardiovascular disease. When the TG level in an individual is higher than 500 mg/dL, the therapeutic goal of lowering TG level takes precedence over the therapeutic goal of lowering LDL-P level. The risk categories and the boundaries dividing them for any biomarker are not limited to those disclosed herein and can be found in the art. Suitable risk categories associated with the second and third risk values determined by cholesterol-absorption biomarkers and cholesterol-synthesis biomarkers include those reference ranges discussed above in Table 1.

Each of cardiovascular risk biomarkers and sterol and/or stanol biomarkers can be associated with a discrete set of risk categories. Combining one category from each biomarker forms a “decision point.” In various exemplary embodiments, the complete set of decision points comprises all possible n-tuples of categories, wherein n is the number of biomarkers evaluated in the decision diagram. This complete set will have m₁×m₂× . . . mₙ possible decision points, wherein mᵢ is the number of categories for biomarker i.

In some cases, several biomarkers used to determine the same risk value can be normalized, or unified to form a single decision point (to be accurate, it is the risk category of the same risk value that form a single decision point). For instance, the cardiovascular risk biomarkers used to determine the first risk value can be considered as a decision point; the cholesterol-absorption biomarkers used to determine the second risk value can be considered as a decision point; and similarly, the cholesterol-synthesis biomarkers used to determine the third risk value can be considered as a decision point. In various exemplary embodiments, a set of decision points for the therapeutic decision diagram comprises all 3 risk categories for each risk value evaluated by various biomarkers; and the therapeutic decision diagram comprises at least 3×3×3 possible decision points.

The conventional cardiovascular risk biomarkers (e.g., LDL-P, ApoB and/or TG) can be used individually or combined to determine whether the patient is in high, medium, or low cardiovascular risk (first risk value). This information can serve as decision points in the decision diagram. Levels of sterol and/or stanol biomarkers can provide information on whether a patient is more of a cholesterol absorber (second risk value, determined by e.g., campesterol, sitosterol, and or cholestanol); a cholesterol synthesizer (third risk value, determined by e.g., desmosterol); both cholesterol absorber and synthesizer; or neither cholesterol absorber nor synthesizer. This information can serve as decision points in the decision diagram.

Every decision point can be associated with a risk or disease state, which is not necessarily unique. That is, one or more decision points can be associated with the same risk or disease state.

Every decision point can also be associated with a particular therapeutic guidance or treatment plan, which is not necessarily unique. That is, one or more decision points may be associated with the same therapy. The association of every possible decision point with one or more therapies can be referred to as a “therapeutic decision diagram.” The therapeutic guidance can be recommended by a doctor or a coach that interprets the data.

Each decision point can be associated with more than one type of information. For example, both risk or disease state and therapy can be indicated by a decision point.

Additional decision points can include an evaluation of a medical history of the subject, for instance, an investigation of the subject's medical record to learn whether the subject is on lipid-modulation medication, etc. If the subject is on lipid-modulation medication, it may be further inquired what lipid medication the subject has been on, e.g., whether the lipid-modulation medication is a drug from statin family, which was used to block cholesterol synthesis, or a drug such as ezetimibe, fenofibrate, supplemental phytosterols or stanols, which was used to reduce cholesterol absorption.

Typically, by the therapeutic decision diagram, the therapeutic guidance at each decision point is not targeted to change the sterol and/or stanol levels in general; rather, the main target is to use these sterol and/or stanol biomarkers as a proxy for cardiovascular risk management by managing lipoprotein goals: e.g., to reach desirable Apo-B and LDL-P levels. The classification of a patient as increased risk (i.e., hyper-) based on their sterol and/or stanol measurement values in plasma may indicate a more aggressive therapy to lower LDL-P and Apo-B levels.

The risk values determined by the sterol and/or stanol biomarkers of both cholesterol absorption (e.g., beta-sitosterol, campesterol, cholestanol) and cholesterol synthesis (e.g., desmosterol) can be used to show the deviations, if any, from the normal reference ranges for each biomarker. Thereafter, a direct indication is the deviation can be adjusted or can be manipulated by drugs such as statins, which can block cholesterol synthesis, or by drugs such as ezetimibe, fenofibrate, supplemental phytosterols or stanols, which can reduce cholesterol absorption.

Additionally, analysis of these cholesterol-synthesis and absorption sterol and/or stanol biomarkers can help identify subjects with phytosterolemia or cerebrotendinous xanthomatosis (CTX). For instance, a significant elevation of cholestanol level can be an indicator of a rare enzyme deficiency called cerebrotendinous xanthomatosis (CTX), which can lead to a specific, xanthomata and CNS neurologic abnormalities.

Analysis of these cholesterol-synthesis and absorption sterol and/or stanol biomarkers can also help identify subjects with ABCG5 and ABCG8 polymorphisms or over-expression of NPC1L1. For instance, a significantly increased absorption biomarker in a subject may indicate loss-of-function mutations in ABCG5 or ABCG8, and an over expression of NPC1L1.

In many scenarios, multiple events and multiple risk factors are convoluted in cholesterol-synthesis and absorption and cardiovascular risk, and it is unclear that simply administering the drug that only functions at one condition would be appropriate without complicating other conditions. The therapeutic decision diagram including different risk categorizations of these sterol and/or stanol biomarkers can assist health practitioners to make more appropriate therapeutic decisions.

The therapeutic decision diagram can help decide when statin monotherapy is or is not suppressing cholesterol synthesis (e.g., help identify statin hypo-responders or stain non-responders) or undesirably increasing sterol absorption. Elevations of plasma non-cholesterol sterols can be powerful biomarkers for cardiovascular risk. For instance, patients with cholesterol hypersynthesis can respond well to statins; whereas patients with hyperabsorptive states (typically associated with menopause or family history of premature CHD) are usually statin hyporesponders.

The therapeutic decision diagram can help identify statin-treated patients who can benefit from the addition of ezetimibe or fibrate; identify statin-intolerant patients who can respond well to fenofibrate/ezetimibe; or identify drug-induced hyperabsorbers (a complication of statin monotherapy) for whom lipid-lowering therapy should include ezetimibe, phytostanol or fibrate.

The therapeutic decision diagram can help identify patients who are on ezetimibe monotherapy that may benefit from addition of a statin or fenofibrate or plant stanol; or patients who are on ezetimibe monotherapy (which can induce cholesterol synthesis) that may benefit from addition of a statin, fibrate or phytostanol.

The therapeutic decision diagram can help identify patients whose initial therapy would benefit from adding ezetimibe to a statin monotherapy. In general, it is undesirable to prescribe statin monotherapy to a patient who hyperabsorbs cholesterol. Also, in general, ezetimibe monotherapy is not used other than treating phytosterolemia.

The therapeutic decision diagram can help monitor patients with underlying hyperabsorptive states who may unknowingly be raising phytosterol levels by using supplemental phytosterols to lower cholesterol. For instance, the health practitioner may have suggested the use of the widely popular, commercially available phytosterols or phytostanol (e.g., Benecol) as a dietary supplement to reduce lower cholesterol levels and manage dyslipidemia. However, if phytosterols are prescribed, the health practitioner needs to monitor markers of phytosterol hyperabsorption (e.g., campesterol, sitosterol, cholestanol): if the levels of these markers elevate, a nonabsorbable phytostanol (Benecol) can be used instead of a phytosterol.

The therapeutic decision diagram can also help explain why certain patients have atherosclerotic events with unremarkable levels of standard lipid concentrations.

After the identification of a risk or disease status of a patient, the treatment algorithms contained in the therapeutic decision diagram can be used accordingly to effectuate the most appropriate treatment strategy according to patent's specific risk or disease status.

The therapeutic guidance may involve a recommendation to increase statin dose administration to the subject, switch to administering a more potent statin to the subject, and/or start administering a drug to the subject to achieve lipoprotein goals.

Alternatively, the therapeutic guidance may involve a recommendation to maintain statin administration to the subject, reduce statin dose administration to the subject, switch to administering a less potent statin to the subject, and/or start administering a drug to the subject to reduce cholesterol absorption.

Alternatively, the therapeutic guidance may involve a recommendation to start administering a low or moderate dose statin, and/or start administering a drug to a subject to achieve lipoprotein goal of therapy as well as to reduce cholesterol absorption.

The drug to be administered in various embodiments includes, but is not limited to, ezetimibe, a form of niacin or nicotinic acid, a form of fibrate, sitostanol, a bile acid sequestrant, and combinations thereof. In the event when the subject is determined to be a hyper-absorber, the drug to be administered should not be a plant sterol, as the subject has already absorbed excess amounts of plant sterol.

The therapeutic guidance may involve a recommendation that the subject does not need to be placed on a medication, or a recommendation to maintain a previous therapeutic guidance that has been provided to the subject.

The therapeutic guidance may further comprise a recommendation to further monitor the level of the cardiovascular risk biomarkers, the level of the cholesterol-absorption sterol and/or stanol biomarkers, and/or the levels of the cholesterol-synthesis sterol and/or stanol biomarkers contained in a biological sample from the subject, to determine the prognosis of the cardiovascular disease, and/or the efficacy of a previous therapeutic guidance that has been provided to the subject.

The therapeutic decision diagram can be used in methods to diagnose, identify or screen subjects that have, do not have or are at risk for cardiovascular disease; to differentially diagnose cardiovascular risk/disease states; to evaluate the severity or changes in severity of cardiovascular risk/disease in a subject; to monitor the efficacy of the therapies for cardiovascular disease on subjects that are undergoing such therapies; to determine or suggest a new therapy or a change in therapy.

Accordingly, one aspect of this invention relates to a method of prognosing, diagnosing, and/or predicting risk of cardiovascular disease in a subject. The method comprises the steps of a) measuring levels of one or more cholesterol-absorption sterol and/or stanol biomarkers contained in a biological sample from the subject, and levels of one or more cholesterol-synthesis sterol and/or stanol biomarkers contained in a biological sample from the subject; b) comparing the levels of the cholesterol-synthesis sterol and/or stanol biomarkers and the cholesterol-absorption sterol and/or stanol biomarkers to reference levels of each corresponding sterol and/or stanol biomarker to determine whether levels of the cholesterol-absorption sterol and/or stanol biomarkers are normal, decreased, or increased, and whether levels of the cholesterol-synthesis sterol and/or stanol biomarkers are normal, decreased, or increased; and c) effectuating a therapeutic plan based on the determination of the levels of the cholesterol-absorption sterol and/or stanol biomarkers and the cholesterol-synthesis sterol and/or stanol biomarkers, and an assessment of whether the subject is at high risk, moderate risk, or low risk of cardiovascular disease.

The assessment of whether the subject is at high risk, moderate risk, or low risk of cardiovascular disease is determined by comparing levels of one or more cardiovascular risk biomarkers contained in a biological sample from the subject to a reference level of each corresponding cardiovascular risk biomarker. The cardiovascular risk biomarkers comprise at least one of low density lipoprotein particle number (LDL-P); LDL cholesterol (LDL-C); apolipoprotein B (ApoB); and triglyceride (TG).

The assessment of whether the subject is at high risk, moderate risk, or low risk of cardiovascular disease can further comprise measuring levels of one or more cardiovascular risk biomarkers comprising at least one of low density lipoprotein particle number (LDL-P), apolipoprotein B (ApoB), or triglyceride (TG) contained in a biological sample from the subject at the first and second time points; and comparing the level of each cardiovascular risk biomarkers at the second time point to the level at the first time point to determine whether the subject is at high risk, moderate risk, or low risk of cardiovascular disease.

The levels of the cardiovascular risk biomarkers, the levels of the cholesterol-absorption sterol and/or stanol biomarkers, and/or the levels of the cholesterol-synthesis sterol and/or stanol biomarkers contained in a biological sample from the subject can be monitored from time to time to determine the prognosis of the cardiovascular disease, and/or the efficacy of a previous therapeutic guidance that has been provided to the subject.

The monitoring steps can comprise measuring levels of one or more cholesterol-absorption sterol and/or stanol biomarkers contained in a biological sample from the subject, and/or levels of one or more cholesterol-synthesis sterol and/or stanol biomarkers contained in a biological sample from the subject at the first and second time points; comparing the level of each absorption biomarker and/or each synthesis biomarker at the second time point to the level of each absorption biomarker and/or each synthesis biomarker at the first time point to determine whether levels of the cholesterol-absorption sterol and/or stanol biomarkers are normal, decreased, or increased, and/or whether levels of the cholesterol-synthesis sterol and/or stanol biomarkers are normal, decreased, or increased; and effectuating a therapeutic plan based on the determination of the levels of the cholesterol-absorption sterol and/or stanol biomarkers and/or the cholesterol-synthesis sterol and/or stanol biomarkers, and an assessment of whether the subject is at high risk, moderate risk, or low risk of cardiovascular disease.

In one embodiment, the therapeutic plan comprises increasing statin dose administration to the subject, switching to administering a more potent statin to the subject, and/or starting administering a drug to the subject to achieve lipoprotein goal of therapy.

Alternatively, the therapeutic plan comprises maintaining statin administration to the subject, reducing statin dose administration to the subject, switching to administering a less potent statin to the subject, and/or starting administering a drug to the subject to reduce cholesterol absorption.

Alternatively, the therapeutic plan comprises starting administering low or moderate dose statin, and/or starting administering a drug to the subject to reduce cholesterol absorption.

Alternatively, the therapeutic plan comprises providing a recommendation that the subject does not need to be placed on a medication, or maintaining a previous therapeutic guidance that has been provided to the subject.

In some embodiments, measurement of levels of a drug used to treat cholesterol imbalance in a subject can be measured in a sample from the subject in order to assess patient truthfulness in taking their prescribed medication in compliance with their treatment guidance. This provides more feedback information to be incorporated into the therapeutic decision diagrams and better direct treatment choices by the health practitioner.

When applying the therapeutic decision diagrams, the health practitioners typically obtain the information necessary to construct decision points in the therapeutic decision diagrams, such as conventional cardiovascular risk biomarkers and/or factors, and cholesterol-synthesis sterols/stanols biomarkers.

For conventional cardiovascular risk category determination, information can be collected, such as patient history (e.g., medical history, family history, and/or pregnancy complication history); symptoms of cardiovascular disease; physical exams (e.g., blood pressure, body mass index, and/or waist size); conventional biomarkers (e.g., LDL-P; ApoB; TG; LDL-C; fasting lipoproteins with Lp-IR score; glycemic/insulin resistance parameters; Apo E genotype; myocardial markers: NT-ProBNP, Galectin-3; coagulation markers; renal markers; and/or homocysteine, omega 3 index, vitamin D).

After the information is obtained, the health practitioners can preliminarily categorize the patient, based on these conventional cardiovascular risk biomarkers and/or factors, into categories of high risk, moderate risk, or low risk of cardiovascular disease. They may also analyze cholesterol-absorption biomarkers and cholesterol-synthesis biomarkers to categorize whether the patient is a hyper-, normal, or hypo (i.e., increased, normal or decreased) in cholesterol-absorption and cholesterol-synthesis (See, for example, Table 1 for the categorization). The health practitioners may also obtain additionally pertinent medical history or treatment history, if needed, based on their discretion. The additional information can be integrated into the essential decision points described above, or can serve as additional decision points. For instance, the information relating to a patient's age, gender, family history, blood pressure, glucose level, cholesterol level, obesity, physical activity frequency, psychosocial factors, obesity, diabetes mellitus, smoking status, alcohol consumption, etc., can be integrated into the decision points to categorize whether the patient has a high risk, moderate risk, or low risk of cardiovascular disease. A patient's medication history, such as lipid-modulation medication, can also be used to construct one or more separate decision points.

The decision diagrams may be particularly useful to health practitioners. For instance, the health practitioners can use the analysis and categorization results for cholesterol-absorption biomarkers and cholesterol-synthesis biomarkers to filter through the therapeutic decision diagrams and decide which particular therapeutic decision diagram to follow. The health practitioners can then compare the patient's information from each decision point with the elements described in the therapeutic decision diagram (e.g., a flow chart), follow and match the patient's information with the exact scenarios described in the therapeutic decision diagram, and provide the corresponding therapeutic guidance proscribed for that specific scenarios matched.

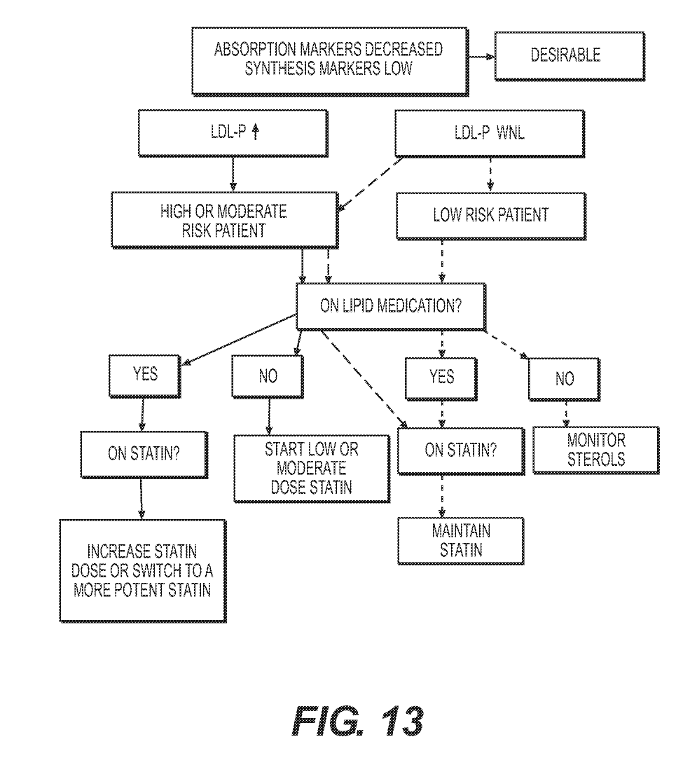

Exemplary clinical interventions based on embodiments described here are detailed in several specific clinical therapeutic decision diagrams in FIGS. 13 to 19. These diagrams are not meant to be limiting, but are set forth to illustrate the various ways these factors can be implemented into decision diagrams.

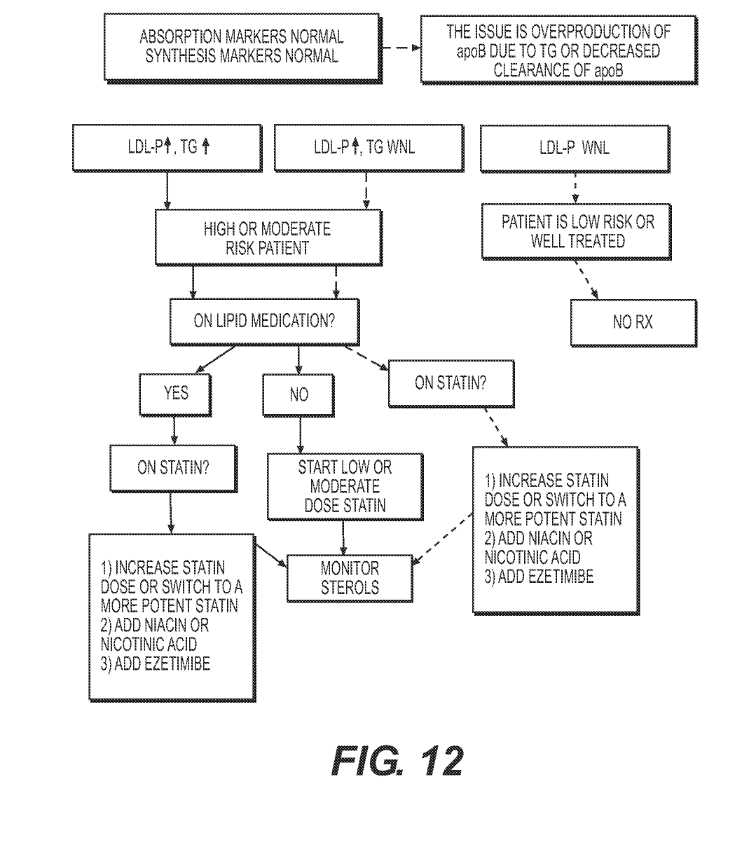

When the analysis results for cholesterol-absorption biomarkers and cholesterol-synthesis biomarkers in a subject show absorption biomarkers (second risk value) as “normal,” and synthesis biomarkers (third risk value) as “normal,” the health practitioners can refer to the therapeutic decision diagram shown in FIG. 12 for therapeutic guidance. If the result for conventional cardiovascular risk biomarkers and/or factors (first risk value; such as LDL-P, and/or TG) is “high risk,” the health practitioners can provide a therapeutic guidance comprising evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject, or switch to administering a more potent statin to the subject, and/or start administering one or more drugs (niacin or nicotinic acid, and/or ezetimibe) to the subject to achieve risk-related LDL-P and apoB goals. If the subject is not on lipid medication, the therapeutic guidance can comprise a recommendation to start administering a low or moderate statin to the subject. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject, or switch to administering a more potent statin to the subject, and/or start administering one or more drugs (niacin or nicotinic acid, and/or ezetimibe) to the subject to achieve risk-related LDL-P and apoB goals. If the first risk value is “low risk,” the therapeutic guidance can comprise a recommendation that the subject does not need to be placed on a medication, or a recommendation to maintain a previous therapeutic guidance that has been provided to the subject.

When the analysis result for absorption and synthesis biomarkers in a subject are “decreased,” and “decreased,” respectively, the health practitioners can refer to the therapeutic decision diagram shown in FIG. 13 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject, or switch to administering a more potent statin to the subject. If the subject is not on a lipid medication, the therapeutic guidance can comprise a recommendation to start administering a low or moderate dose statin to the subject. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. If the subject is not on a lipid medication, the therapeutic guidance can comprise a recommendation to monitor the levels of the cholesterol synthesis and absorption sterols and/or stanol biomarkers.

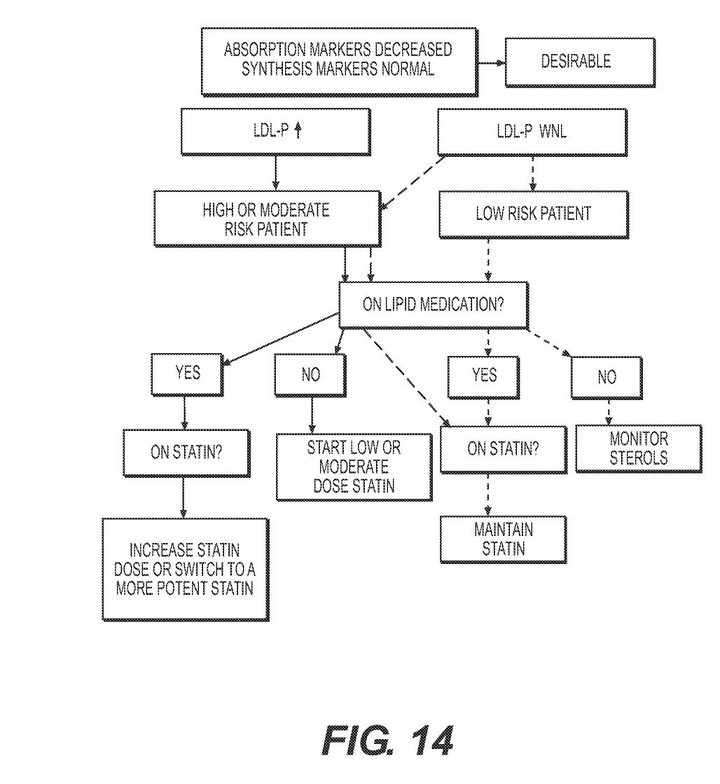

When the analysis results for absorption and synthesis biomarkers in a subject are “decreased,” and “normal,” respectively, the health practitioners can refer to the therapeutic decision diagram shown in FIG. 14 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject, or switch to administering a more potent statin to the subject. If the subject is not on a lipid medication, the therapeutic guidance can comprise a recommendation to start administering a low or moderate dose statin to the subject. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. If the subject is not on a lipid medication, the therapeutic guidance can comprise a recommendation to monitor the levels of the cholesterol synthesis and absorption sterols and/or stanol biomarkers.

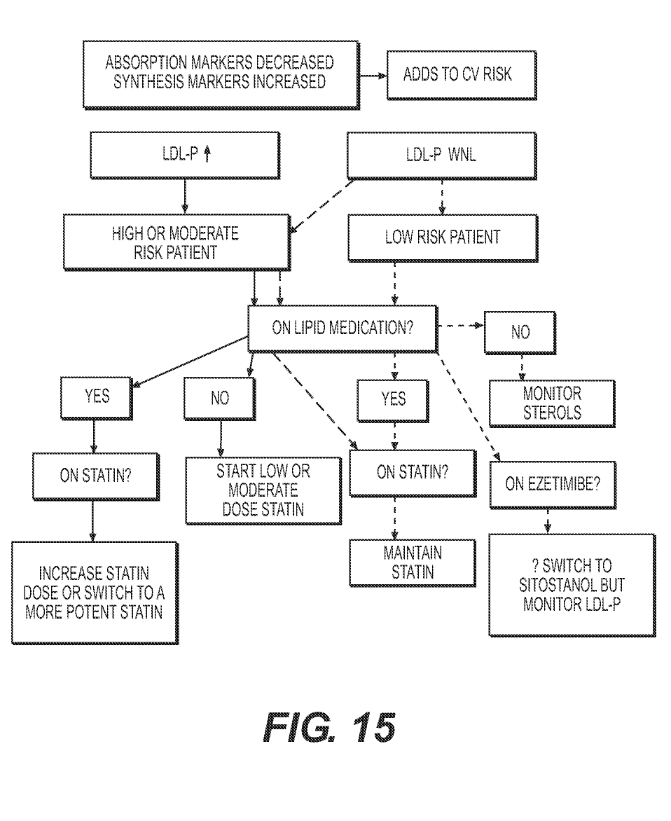

When the analysis results for absorption and synthesis biomarkers in a subject are “decreased,” and “increased,” respectively, the health practitioners can refer to the therapeutic decision diagram shown in FIG. 15 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject, or switch to administering a more potent statin to the subject. If the subject is not on a lipid medication, the therapeutic guidance can comprise a recommendation to start administering a low or moderate dose statin to the subject. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. If the subject is on ezetimibe medication, the therapeutic guidance can comprise a recommendation to switch to administering sitostanol, and at the same time monitoring the level of LDL-P and/or ApoB markers. If the subject is not on a lipid medication, the therapeutic guidance can comprise a recommendation to monitor the levels of the cholesterol synthesis and absorption sterols and/or stanol biomarkers.

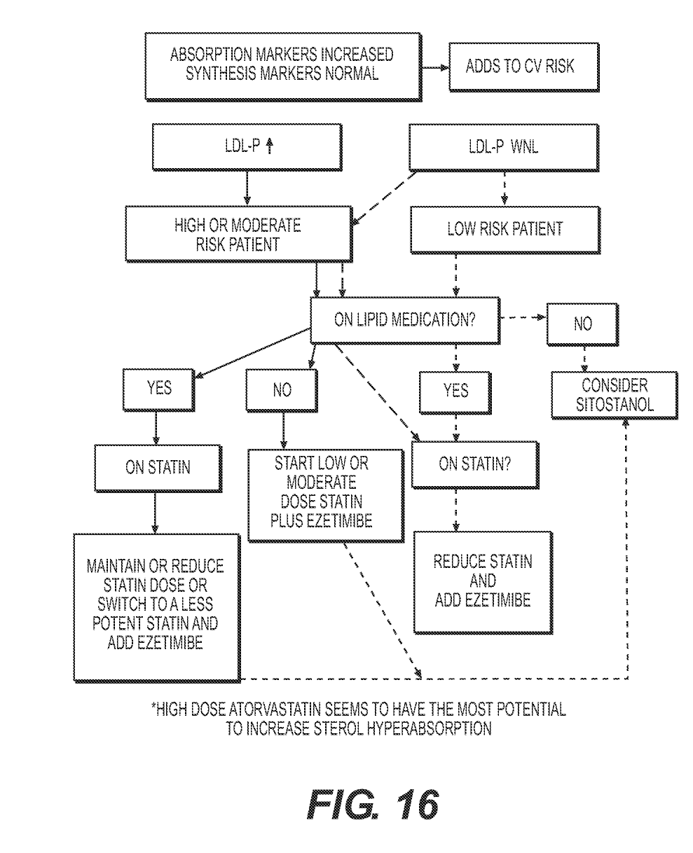

When the analysis results for absorption and synthesis biomarkers in a subject are “increased,” and “normal,” respectively, the health practitioners can refer to the therapeutic decision diagram shown in FIG. 16 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintaining or reducing statin dose administration to the subject or switch to administering a less potent statin to the subject, and start administering ezetimibe. The therapeutic guidance may further comprise a recommendation to starting considering or administering sitostanol. If the subject is not on a lipid medication, the therapeutic guidance can comprise a recommendation to start administering a low or moderate dose statin to the subject, and start administering ezetimibe. The therapeutic guidance may further comprise a recommendation to starting considering or administering sitostanol. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to reduce statin dose administration to the subject, and start adding ezetimibe to the current medication. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to reduce statin dose administration to the subject, and start adding ezetimibe to the current medication. If the subject is not on a lipid medication, the therapeutic guidance can comprise a recommendation to starting considering or administering sitostanol.

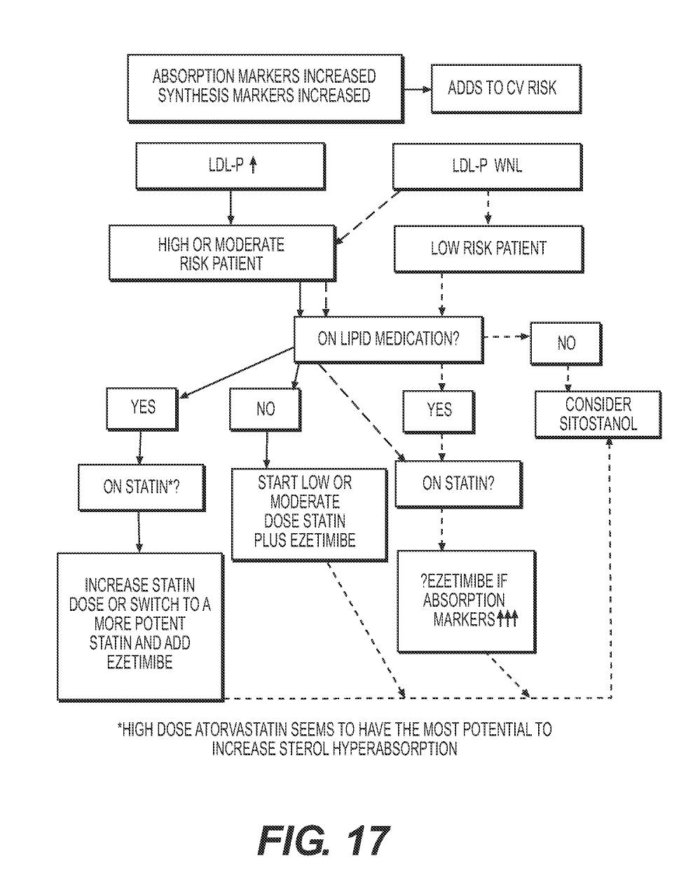

When the analysis results for absorption and synthesis biomarkers in a subject are “increased,” and “increased,” respectively, the health practitioners can refer to the therapeutic decision diagram shown in FIG. 17 for therapeutic guidance. In various embodiments, if the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject or switch to administering a more potent statin to the subject, and start adding ezetimibe to the current medication. The therapeutic guidance can further comprise a recommendation to starting considering or administering sitostanol. If the subject is not on a lipid medication, the therapeutic guidance can comprise a recommendation to start administering a low or moderate statin to the subject, and start adding ezetimibe to the current medication. The therapeutic guidance may further comprise a recommendation to starting considering or administering sitostanol. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to start administering ezetimibe, if the absorption markers have increased significantly. The therapeutic guidance may further comprise a recommendation to starting considering or administering sitostanol. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to start administering ezetimibe, if the absorption markers have increased significantly. If the subject is not on a lipid medication, the therapeutic guidance can comprise a recommendation to starting considering or administering sitostanol.

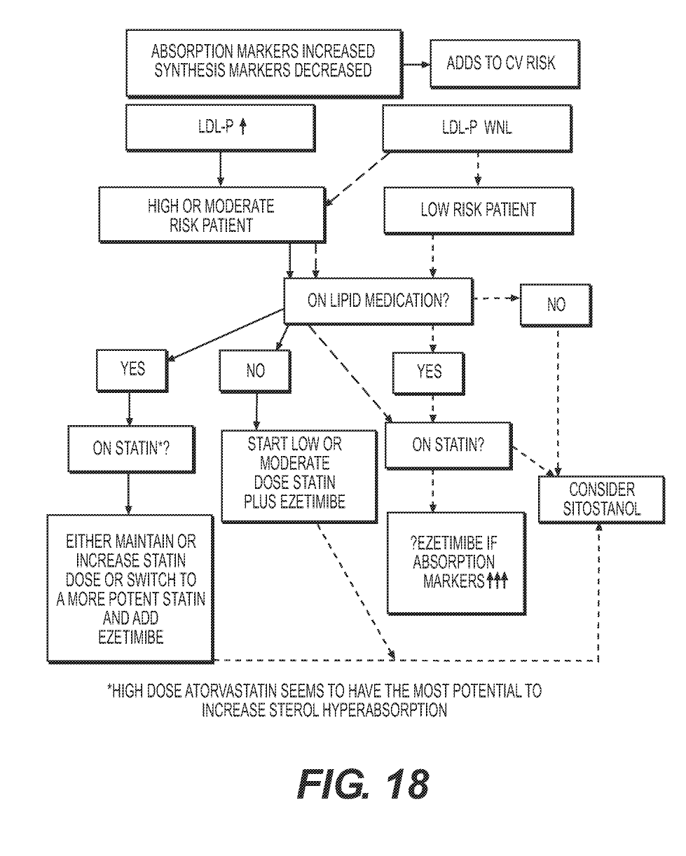

When the analysis results for absorption and synthesis biomarkers in a subject are “increased,” and “decreased,” respectively, the health practitioners can refer to the therapeutic decision diagram shown in FIG. 18 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to either maintain or increase statin dose administration to the subject or switch to administering a more potent statin to the subject, and start administering ezetimibe. The therapeutic guidance may further comprise a recommendation to starting considering or administering sitostanol. If the subject is not on a lipid medication, the therapeutic guidance can comprise a recommendation to start administering a low or moderate statin to the subject, and start adding ezetimibe to the current medication. The therapeutic guidance may further comprise a recommendation to starting considering or administering sitostanol. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to start administering ezetimibe, if the absorption markers have increased significantly. The therapeutic guidance may further comprise a recommendation to starting considering or administering sitostanol. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to start administering ezetimibe, if the absorption markers have increased significantly. The therapeutic guidance may further comprise a recommendation to starting considering or administering sitostanol. If the subject is not on a lipid medication, the therapeutic guidance can comprise a recommendation to starting considering or administering sitostanol.

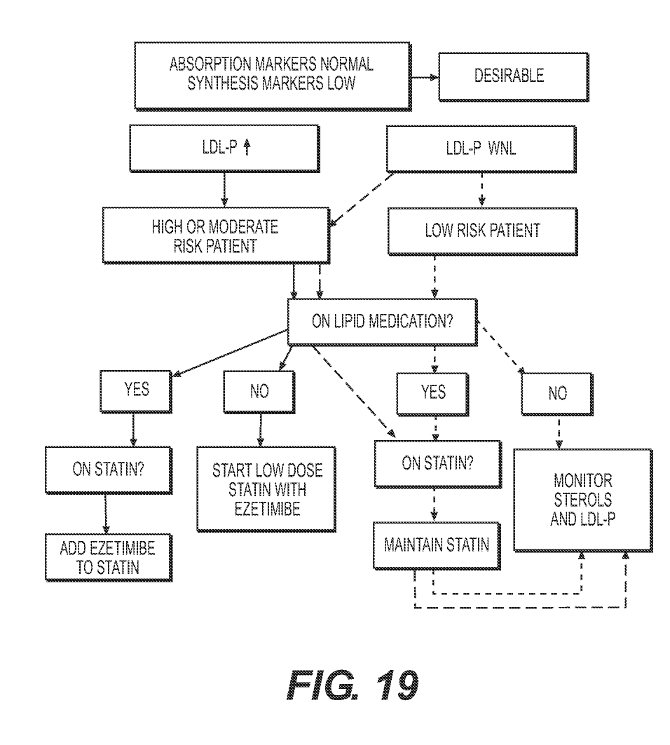

When the analysis results for absorption and synthesis biomarkers in a subject are “normal,” and “decreased,” respectively, the health practitioners can refer to the therapeutic decision diagram shown in FIG. 19 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to add ezetimibe to the current statin medication. If the subject is not currently on a lipid medication, the therapeutic guidance can comprise a recommendation to start administering a low dose statin with ezetimibe to the subject. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. The therapeutic guidance may further comprise a recommendation to monitor the levels of the cholesterol synthesis and absorption sterols and/or stanol biomarkers and the level of LDL-P marker. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to maintain statin administration to the subject. The therapeutic guidance may further comprise a recommendation to monitor the levels of the cholesterol synthesis and absorption sterols and/or stanol biomarkers and the level of LDL-P marker. If the subject is not on a lipid medication, the therapeutic guidance can comprise a recommendation to monitor the levels of the cholesterol synthesis and absorption sterols and/or stanol biomarkers and the level of LDL-P marker.

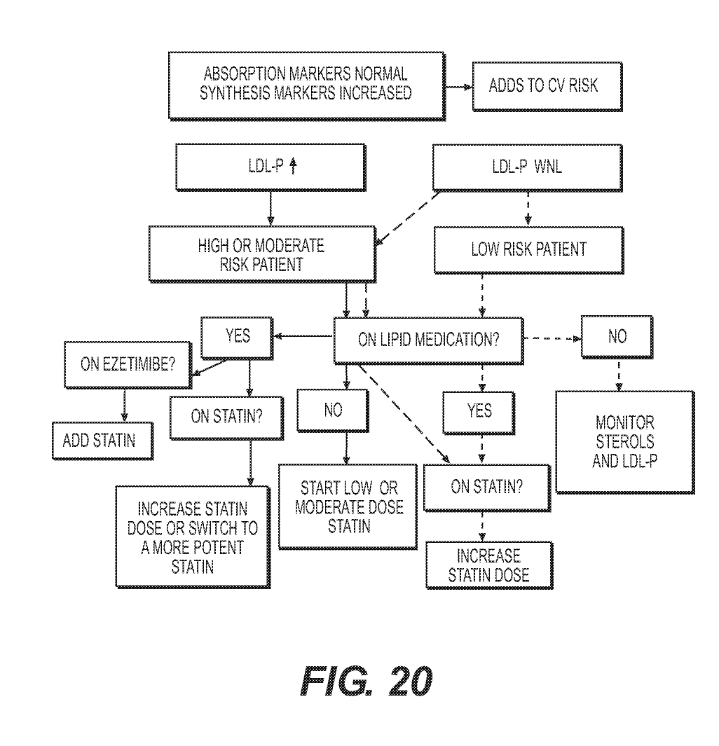

When the analysis results for absorption and synthesis biomarkers in a subject are “normal,” and “increased,” respectively, the health practitioners can refer to the therapeutic decision diagram shown in FIG. 20 for therapeutic guidance. If the first risk value is “high risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin dose administration to the subject, or switch to administering a more potent statin to the subject. If the subject is not on statin medication, but on ezetimibe medication, the therapeutic guidance can comprise a recommendation to start adding statin to the current medication. If the subject is not currently on a lipid medication, the therapeutic guidance can comprise a recommendation to start administering a low or moderate dose statin to the subject. If the first risk value is “moderate risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin administration to the subject. If the first risk value is “low risk,” the therapeutic guidance can comprise evaluation of a medical history of the subject, for instance, whether the subject has been or is currently on a lipid-modulating medication, such as a drug from the statin family. If the subject is on statin medication, the therapeutic guidance can comprise a recommendation to increase statin administration to the subject. If the subject is not on a lipid medication, the therapeutic guidance can comprise a recommendation to monitor the levels of the cholesterol synthesis and absorption sterols and/or stanol biomarkers and the level of LDL-P marker.

Of course, a health practitioner is not bound to follow all or any of the recommendations set forth in the decision diagrams. Nor is a health practitioner required to choose a particular diagram to follow. Discretion and medical knowledge will always play a key role in any decision or recommendation a health practitioner makes.

After each scenario, the therapeutic guidance can comprise a recommendation to monitor the level of the cardiovascular risk biomarkers, and/or the levels of the cholesterol synthesis and absorption sterols/stanols biomarkers, to determine the prognosis of the cardiovascular risk/disease, and/or the efficacy of the previous therapeutic guidance that has been provided to the subject.

When making therapeutic guidance using the clinical therapeutic decision diagram, the following additional factors may be considered.

The phytosterolemia disease is not common, and levels of sitosterol and/or campesterol may be associated with phytosterolemia disease when they reach the range of 100-300 μg/mL. Although elevations of phytosterols may be associated with cardiovascular risk, health practitioners may not directly use phytosterol levels as causal of atherosclerosis.

Phytosterols used as nutritional supplements can increase phytosterol levels in hyperabsorbers and thus are not recommended in those situations.

Phytostanols such as sitostanol (Benecol) may not be absorbed to an appreciable degree and can be used in patients with hyperabsorption of sterols.

When LDL-P (apoB) are elevated while the levels of cholesterol synthesis and absorption sterols and/or stanol biomarkers are normal, there may be a problem with overproduction (typically relate to TG abnormalities) or decreased clearance of apoB particles (e.g., LDL receptor issues, defective apoB, and/or PCSK9 gain of function issues).

With respect to treatment decisions in persons with sterol synthesis/absorption abnormalities, the following factors may be taken into account: i) unless phytosterolemia is present or the patient is in the very high risk category, the therapy goal is typically to achieve desirable levels of lipoproteins (e.g., LDL-P or apoB), not levels of cholesterol synthesis and absorption sterols and/or stanol biomarkers per se; ii) the higher the overall risk of the patient determined from various sterols and/or stanol biomarkers levels, the more aggressive therapeutic goal of apoB (LDL-P) may be used; iii) unless TG levels are >500 mg/dL, statins are typically initially used to lower LDL-P levels; and iv) if the patient is a hyperabsorber despite of elevated LDL-P levels, statin/ezetimibe may be administered.

Clinical therapeutic decision diagrams to guide treatment can be provided by any form, such as a computer program, world wide web page, or cards. Any suitable presentation, such as pictures, graphs, schemes, flow charts, animations, depictions or exemplifications may be used in the therapeutic decision diagram. For example, the therapeutic decision diagrams can be represented as schemes of flow charts, so that health practitioners can look, compare and match the subject's risk values with the risk category described in the charts, and provide corresponding therapeutic guidance from the therapeutic decision diagram.

The methods described herein may be implemented using any device capable of implementing the methods. Examples of devices that may be used include but are not limited to electronic computational devices, including computers of all types. When the methods are implemented in a computer, the computer program that may be used to configure the computer to carry out the steps of the methods may be contained in any computer readable medium capable of containing the computer program. For example, the decision points and parameters of the therapeutic decision diagrams can be formatted in a computer program (e.g., a software form). The health practitioner or the subject can enter measured results of different biomarkers, as well as a subject's information based on a series of questions around medical history and health results. The computer program then calculates and presents the recommended therapeutic guidance(s) to the health practitioner/or the subject. The health practitioner can then act on the recommendation if the results are not provided directly to the subject. This computer program, including the reference levels of different biomarkers and cardiovascular factors, may be contained in a computer readable medium. Examples of computer readable medium that may be used include but are not limited to diskettes, CD-ROMs, DVDs, ROM, RAM, and other memory and computer storage devices.

The computer program that may be used to configure the computer to carry out the steps of the methods may also be provided over an electronic network, for example, over the internet, world wide web, an intranet, or other network. It can also be downloaded to a computer or other electronic device such as a laptop, smart-phone, ipad, or the IT network in a provider's office. An exemplary application that carries out the steps of the methods downloadable to a computer or a smart-phone (such as iphone or ipad) has been described in details in U.S. Provisional Application No. 61/747,505, entitled, “Biomarker Bliki,” filed Dec. 31, 2012, which is herein incorporated by reference in its entirety.

### REFERENCES

1. MARANGONI, “Poli A. Phytosterols and Cardiovascular Health” Pharmacol. Res. 61:193-199 (2010)
2. CALPE-BERDIE et al., “New Insights into the Molecular Actions of Plant Sterols and Stanols in Cholesterol Metabolism” Atherosclerosis 203(1):18-31 (2009)
3. FAHY et al., “A comprehensive Classification System for Lipids” J. Lipid Res. 46(5): 839-61 (2005)
4. CHAN et al., “Plasma Concentrations of Plant Sterols: Physiology and Relationship with Coronary Heart Disease” Nutr. Rev. 64:385-402 (2006)
5. MOREAU et al., “Phytosterols, phytostanols, and their conjugates in foods: structural diversity, quantitative analysis, and health-promoting uses. Prog. Lipids Res. 2002; 41(6):457-500.
6. DE JONG et al., “Metabolic Effects of Plant Sterols and Stanols” J. Nutr. Biochem. 14:362-9 (2003)
7. FRANSEN et al., “Customary Use of Plant Sterol and Plant Stanol Enriched Margarine is Associated with Changes in Serum Plant Sterol and Stanol Concentrations in Humans” J. Nutr. 137:1301-1306 (2007)
8. DAYSPRING, “Phytosterolemia: Synthesis, Absorption, Trafficking and Excretion of Cholesterol and Noncholesterol Sterols” Therapeutic Lipidology (Chapter 14). Davidson MH, Toth PP, and Maki KC. Editors 2008 Humana Press Totowa, N.J.
9. PATEL & THOMPSON, “Phytosterols and Vascular Disease” Atherosclerosis 186(1):12-9 (2006)
10. PLAT & MENSINK, “Plant Stanol and Sterol Esters in the Control of Blood Cholesterol Levels: Mechanism and Safety Aspects” Am. J. Cardiol. 96:15 D-22D (2005)
11. SUDHOP & VON BERGMANN, “Sitosterolemia—A Rare Disease. Are Elevated Plant Sterols An Additional Risk Factor?” Z. Kardiol. 93(12):921-8 (2004)
12. SHIBATA & GLASS, “Macrophages, Oxysterols and Atherosclerosis” Circ. J. 74(10):2045-51 (2010)
13. CATAPANO et al., “ESC/EAS Guidelines for the Management of Dyslipidaemias: The Task Force for the Management of Dyslipidaemias of the European Society of Cardiology (ESC) and the European Atherosclerosis Society (EAS)” Atherosclerosis 217S:S1-S44 (2011)
14. SEHAYEK et al., “U-shape Relationship Between Change in Dietary Cholesterol Absorption and Plasma Lipoprotein Responsiveness and Evidence for Extreme Interindividual Variation in Dietary Cholesterol Absorption in Humans” J. Lipid Res. 39(12):2415-22 (1998)
15. KEREN & FALIK-ZACCAI, “Cerebrotendinous Xanthomatosis (CTX): A Treatable Lipid Storage Disease” Pediatr. Endocrinol. Rev. 7(1):6-11 (2009)
16. MATTHAN et al. “Alterations in Cholesterol Absorption/Synthesis Markers Characterize Framingham Offspring Study Participants with CHD” J. Lipid Res. 50(9):1927-35 (2009)
17. ASSMANN et al., “Plasma Sitosterol Elevations are Associated with an Increased Incidence of Coronary Events in Men: Results of a Nested Case-Control Analysis of the Prospective Cardiovascular Munster (PROCAM) Study” Nutr. Metab. Cardiovasc. Dis. 16(1):13-21 (2006)
18. CALPE-BERDIEL et al., “Increased Plasma Levels of Plant Sterols and Atherosclerosis: A Controversial Issue” Curr. Athero. Rep. 11:391-398 (2009)
19. SUDHOP et al., “Serum Plant Sterols as a Potential Risk Factor for Coronary Heart Disease” Metabolis. 51(12):1519-1521 (2002)
20. MIETTINEN et al., “Relation of Non-Cholesterol Sterols to Coronary Risk Factors and Carotid Intima-Media Thickness: The Cardiovascular Risk in Young Finns Study” Atherosclerosis 209(2):592-7 (2010)
21. STRANDBERG et al., “Serum Plant and Other Noncholesterol Sterols, Cholesterol Metabolism and 22-Year Mortality Among Middle-Aged Men” Atherosclerosis 210:282-287 (2010)
22. SILBERNAGEL et al., “The Relationships of Cholesterol Metabolism and Plasma Plant Sterols with the Severity of Coronary Artery Disease” J. Lipid Res. 50(2):334-41 (2009)
23. SILBERNAGAL et al., “The Associations of Cholesterol Metabolism and Plasma Plant Sterols with All-Cause and Cardiovascular Mortality” J. Lipid Res. 51:2384-2393 (2010)
24. WILUND et al., “No Association Between Plasma Levels of Plant Sterols and Atherosclerosis In Mice and Men” Arterioscler. Thromb. Vasc. Biol. 24(12):2326-32 (2004)
25. STRANDBERG et al., “Cholesterol and Glucose Metabolism and Recurrent Cardiovascular Events Among the Elderly: A Prospective Study” J. Am. Coll. Cardiol. 48:708-714 (2006)
26. IZAR et al., “Phytosterols and Phytosterolemia: Gene-Diet Interactions” Genes Nutr. 6(1):17-26 (2011)
27. JAKULJ et al., “ABCG5/G8 Polymorphisms and Markers of Cholesterol Metabolism: Systematic Review and Meta-Analysis” J. Lipid Res. 51(10):3016-23 (2010)
28. GYLLINGA et al., “The Metabolism of Plant Sterols is Disturbed in Postmenopausal Women with Coronary Artery Disease” Metabolism Clinical and Experimental 58:401-407 (2009)
29. JASINSKA et al., “Statins: A New Insight Into Their Mechanisms of Action and Consequent Pleiotropic Effects” Pharmacol. Rep. 59(5):483-99 (2007)
30. GYLLING & MIETTINEN, “Cholesterol Absorption and Lipoprotein Metabolism in Type Ii Diabetes Mellitus With and Without Coronary Artery Disease” Atherosclerosis 126:325-332 (1996)
31. HAGBERG et al., “APO E Gene and Gene-Environment Effects on Plasma Lipoprotein-Lipid Levels” Physiol. Genomics 4(2):101-108 (2000)
32. MIETTINEN et al., “Plant Sterols In Serum and In Atherosclerotic Plaques of Patients Undergoing Carotid Endarterectomy” J. Am. Coll. Cardiol. 45(11):1794-801 (2005)
33. VALASEK et al., “Fenofibrate Reduces Intestinal Cholesterol Absorption Via PPARalpha-Dependent Modulation of NPC1L1 Expression in Mouse” J. Lipid Res. 48(12):2725-35 (2007)
34. SUDHOP et al., “Inhibition of Intestinal Cholesterol Absorption By Ezetimibe in Humans” Circulation 106: 1943-1948 (2002)
35. LAW, “Plant Sterol and Stanol Margarines and Health” BMJ:320: 861-864 (2000)
36. WESTSTRATE & MEIJER, “Plant Sterol-Enriched Margarines and Reduction of Plasma Total- and LDL-Cholesterol Concentrations in Normocholesterolaemic and Mildly Hypercholesterolaemic Subjects” Eur. J. Clin. Nutr. 52:334-343.
37. MIETTINEN et al., “Baseline Serum Cholestanol as Predictor of Recurrent Coronary Events in Subgroup of Scandinavian Simvastatin Survival Study” Finnish 4S Investigators. BMJ:316(7138):1127-30 (1998)
38. ASSMANN et al., “Effects of Ezetimibe, Simvastatin, Atorvastatin, and Ezetimibe-Statin Therapies on On-Cholesterol Sterols in Patients with Primary Hypercholesterolemia” Curr. Med. Res. Opin. 24(1):249-59 (2008)
39. DAVIDSON, “Therapies Targeting Exogenous Cholesterol Uptake: New Insights and Controversies” Curr. Atheroscler. Rep.13(1):95-100 (2011)
40. KASTELEIN et al., “Simvastatin With or Without Ezetimibe in Familial Hypercholesterolemia” N. Engl. J. Med. 358: 1431-1443 (2008)
41. BALLANTYNE et al., “Efficacy and Safety of Rosuvastatin 40 mg Alone or in Combination with Ezetimibe in Patients At High Risk of Cardiovascular Disease (results from the EXPLORER study)” Am. J. Cardiol. 99: 673-680 (2007)
42. NUTESCU & SHAPIRO, “Ezetimibe: A Selective Cholesterol Absorption Inhibitor” Pharmacotherapy 23(11):1463-74 (2003)
43. THOMPSON, “Additive Effects of Plant Sterol and Stanol Esters to Statin Therapy” Am. J. Cardiol. 86:46-52 (2000)

What is claimed is:

1. A therapeutic decision diagram that determines what therapeutic guidance for achieving lipoprotein goals, if any, should be provided to a subject having various levels of cardiovascular disease risk, said therapeutic decision diagram providing a therapeutic guidance to the subject based on risk values comprising:
(i) a first risk value determined by levels of one or more cardiovascular risk biomarkers contained in a biological sample from the subject, said cardiovascular risk biomarkers comprising at least one of low density lipoprotein particle number (LDL-P), apolipoprotein B (ApoB), or triglyceride (TG); and
(ii) a second risk value determined by levels of one or more cholesterol-absorption sterol and/or stanol biomarkers contained in a biological sample from the subject, and
(iii) a third risk value determined by levels of one or more cholesterol-synthesis sterol and/or stanol biomarkers contained in a biological sample from the subject.

2. The therapeutic decision diagram of claim 1, wherein the first risk value comprises high risk, moderate risk, or low risk of cardiovascular disease, when the levels of the cardiovascular risk biomarkers are compared to a reference level of each corresponding cardiovascular risk biomarker.

3. The therapeutic decision diagram of claim 1, wherein the second risk value comprises normal, decreased, or increased risk of cardiovascular disease, when the levels of the cholesterol-absorption sterol and/or stanol biomarkers are compared to a reference level of each corresponding sterol and/or stanol biomarker.

4. The therapeutic decision diagram of claim 3, wherein the cholesterol-absorption sterol and/or stanol biomarker is selected from the group consisting of campesterol, sitosterol, and cholestanol.

5. The therapeutic decision diagram of claim 1, wherein the third risk value comprises normal, decreased, or increased risk of cardiovascular disease, when the levels of the cholesterol-synthesis sterol and/or stanol biomarkers are compared to a reference level of each corresponding sterol and/or stanol biomarker.

6. The therapeutic decision diagram of claim 5, wherein the cholesterol-synthesis sterol and/or stanol biomarker comprises at least desmosterol.

7. The therapeutic decision diagram of claim 1, wherein the determination of therapeutic guidance includes evaluation of a medical history of the subject.

8. The therapeutic decision diagram of claim 1, wherein the medical history of the subject includes a record whether the subject has been or is currently on a lipid-modulating medication.

9. The therapeutic decision diagram of claim 8, wherein the lipid-modulating medication is a drug from the statin family.

10. The therapeutic decision diagram of claim 9, wherein the therapeutic guidance comprises a recommendation to increase statin dose administration to the subject, switch to administering a more potent statin to the subject, and/or start administering a drug to the subject to achieve lipoprotein goals.

11. The therapeutic decision diagram of claim 10, wherein the drug to achieve lipoprotein goal of therapy is selected from the group consisting of ezetimibe, a form of niacin, a form of fibrate, sitostanol, and combinations thereof.

12. The therapeutic decision diagram of claim 10, wherein the therapeutic guidance further comprises a recommendation not to administer a plant sterol.

13. The therapeutic decision diagram of claim 9, wherein the therapeutic guidance comprises a recommendation to maintain statin administration to the subject, reduce statin dose administration to the subject, switch to administering a less potent statin to the subject, and/or start administering a drug to the subject to reduce cholesterol absorption.

14. The therapeutic decision diagram of claim 13, wherein the drug to reduce cholesterol absorption is selected from the group consisting of ezetimibe, a form of fibrate, sitostanol, and combinations thereof.

15. The therapeutic decision diagram of claim 13, wherein the therapeutic guidance further comprises a recommendation not to administer a plant sterol.

16. The therapeutic decision diagram of claim 1, wherein the therapeutic guidance comprises a recommendation to starting administering low or moderate dose statin, and/or start administering a drug to the subject to achieve lipoprotein goal of therapy as well as to reduce cholesterol absorption.

17. The therapeutic decision diagram of claim 16, wherein the drug is selected from the group consisting of ezetimibe, a form of fibrate, sitostanol, and combinations thereof.

18. The therapeutic decision diagram of claim 16, wherein the therapeutic guidance further comprises a recommendation not to administer a plant sterol.

19. The therapeutic decision diagram of claim 1, wherein the therapeutic guidance comprises a recommendation that the subject does not need to be placed on a medication, or a recommendation to maintain a previous therapeutic guidance that has been provided to the subject.

20. The therapeutic decision diagram of claim 1, wherein the therapeutic guidance comprises a recommendation to further monitor the level of the cardiovascular risk biomarkers, the level of the cholesterol-absorption sterol and/or stanol biomarkers, and/or the levels of the cholesterol-synthesis sterol and/or stanol biomarkers contained in a biological sample from the subject, to determine the prognosis of the cardiovascular disease, and/or the efficacy of a previous therapeutic guidance that has been provided to the subject.

21. A method of prognosing, diagnosing, and/or predicting risk of cardiovascular disease in a subject, comprising:
a). measuring levels of one or more cholesterol-absorption sterol and/or stanol biomarkers contained in a biological sample from the subject, and levels of one or more cholesterol-synthesis sterol and/or stanol biomarkers contained in a biological sample from the subject;
b). comparing the levels of the cholesterol-synthesis sterol and/or stanol biomarkers and the cholesterol-absorption sterol and/or stanol biomarkers to reference levels of each corresponding sterol and/or stanol biomarker to determine whether levels of the cholesterol-absorption sterol and/or stanol biomarkers are normal, decreased, or increased, and whether levels of the cholesterol-synthesis sterol and/or stanol biomarkers are normal, decreased, or increased; and
c). effectuating a therapeutic plan based on said determination of the levels of the cholesterol-absorption sterol and/or stanol biomarkers and the cholesterol-synthesis sterol and/or stanol biomarkers, and an assessment of whether the subject is at high risk, moderate risk, or low risk of cardiovascular disease.

22. The method of claim 21, wherein the assessment of whether the subject is at high risk, moderate risk, or low risk of cardiovascular disease is determined by comparing levels of one or more cardiovascular risk biomarkers contained in a biological sample from the subject to a reference level of each corresponding cardiovascular risk biomarker, said cardiovascular risk biomarkers comprising at least one of low density lipoprotein particle number (LDL-P); LDL cholesterol (LDL-C); apolipoprotein B (ApoB), triglyceride (TG).

23. The method of claim 21, wherein the cholesterol-absorption sterol and/or stanol biomarker is selected from the group consisting of campesterol, sitosterol, or cholestanol.

24. The method of claim 21, wherein the cholesterol-synthesis biomarker comprises at least desmosterol.

25. The method of claim 21, wherein the effectuating the therapeutic plan comprises increasing statin dose administration to the subject, switching to administering a more potent statin to the subject, and/or starting administering a drug to the subject to achieve lipoprotein goal of therapy.

26. The method of claim 21, wherein the effectuating the therapeutic plan comprises maintaining statin administration to the subject, reducing statin dose administration to the subject, switching to administering a less potent statin to the subject, and/or starting administering a drug to the subject to reduce cholesterol absorption.

27. The method of claim 21, wherein the effectuating the therapeutic plan comprises starting administering low or moderate dose statin, and/or starting administering a drug to the subject to reduce cholesterol absorption.

28. The method of claim 21, wherein the effectuating the therapeutic plan comprises providing a recommendation that the subject does not need to be placed on a medication, or maintaining a previous therapeutic guidance that has been provided to the subject.

29. The method of claim 21, further comprising monitoring the levels of the cardiovascular risk biomarkers, the levels of the cholesterol-absorption sterol and/or stanol biomarkers, and/or the levels of the cholesterol-synthesis sterol and/or stanol biomarkers contained in a biological sample from the subject to determine the prognosis of the cardiovascular disease, and/or the efficacy of a previous therapeutic guidance that has been provided to the subject.

30. The method of claim 29, wherein the monitoring comprises:
measuring levels of one or more cholesterol-absorption sterol and/or stanol biomarkers contained in a biological sample from the subject, and/or levels of one or more cholesterol-synthesis sterol and/or stanol biomarkers contained in a biological sample from the subject at the first and second time points;
comparing the level of each absorption biomarker and/or each synthesis biomarker at the second time point to the level of each absorption biomarker and/or each synthesis biomarker at the first time point to determine whether levels of the cholesterol-absorption sterol and/or stanol biomarkers are normal, decreased, or increased, and/or whether levels of the cholesterol-synthesis sterol and/or stanol biomarkers are normal, decreased, or increased; and
effectuating a therapeutic plan based on said determination of the levels of the cholesterol-absorption sterol and/or stanol biomarkers and/or the cholesterol-synthesis sterol and/or stanol biomarkers, and an assessment of whether the subject is at high risk, moderate risk, or low risk of cardiovascular disease.

31. The method of claim 30, wherein the assessment of whether the subject is at high risk, moderate risk, or low risk of cardiovascular disease comprises:
measuring levels of one or more cardiovascular risk biomarkers comprising at least one of low density lipoprotein particle number (LDL-P), apolipoprotein B (ApoB), or triglyceride (TG) contained in a biological sample from the subject at the first and second time points; and
comparing the level of each cardiovascular risk biomarkers at the second time point to the level at the first time point to determine whether the subject is at high risk, moderate risk, or low risk of cardiovascular disease.
